(19) United States
(12) Patent Application Publication
(10) Pub. No.: US 2023/0149020 A1
(43) Pub. Date: May 18, 2023

(54) DEVICES AND METHODS FOR AT LEAST PARTIALLY OCCLUDING A BODILY LUMEN

(71) Applicant: Endovision Foundation, San Francisco, CA (US)

(72) Inventors: Kenneth F. Binmoeller, San Francisco, CA (US); Hoang Phan, Milpitas, CA (US); John P. Lunsford, San Carlos, CA (US); Fiona Sander, Los Altos Hills, CA (US)

(21) Appl. No.: 17/907,002

(22) PCT Filed: Mar. 19, 2021
(86) PCT No.: PCT/US2021/023150

§ 371 Date: Sep. 22, 2022

Related U.S. Application Data
(60) Provisional application No. 62/993,192, filed on Mar. 23, 2020.

(51) Int. Cl. A61B 17/12

(52) U.S. Cl. CPC A61B 17/1204; A61B 17/12136; A61B 2017/00818

(57) ABSTRACT

Described herein are devices and related methods for occluding at least a portion of a bodily lumen during a procedure. In some embodiments, the device includes an inner shaft defining a lumen therethrough and a first aperture; and an outer shaft including a flexible member and defining a lumen therethrough and a second aperture positioned proximally on the outer shaft relative to the flexible member. The inner shaft extends through the lumen of the outer shaft, such that at least a portion of the outer shaft is axially translatable toward and away from the distal portion of the inner shaft. The flexible member is movable between an unexpanded configuration and an expanded configuration when at least a portion of the outer shaft is translated axially toward or away from the distal portion of the inner shaft. In some embodiments, the device and methods are used for endoscopy procedures.

### CROSS-REFERENCE TO RELATED APPLICATIONS

This application claims the priority benefit of U.S. Provisional Patent Application Ser. No. 62/993,192, filed Mar. 23, 2020, the contents of which are herein incorporated by reference in their entirety.

### TECHNICAL FIELD

This disclosure relates generally to the field of endoscopy, and more specifically to the field of endoscopic ultrasound. Described herein are systems and methods for at least partially occluding a bodily lumen during a procedure.

### BACKGROUND

Currently, endoscopic ultrasound guided procedures, for example entero-enterostomy (includes gastro-enterostomy), are limited by the quality of the ultrasound imaging and lumen distensibility of the targeted portion of bowel. Portions of the gastrointestinal tract are flooded with liquid so that imaging can be performed. However, liquid rapidly passes through the gastrointestinal (GI) tract, limiting the available time for imaging and distensibility of the lumen for targeted intervention. Large quantities of liquid can be infused in an effort to counter the egress, but excessive liquid infusion (more than 500 ml) into the bowel may lead to metabolic disturbances. As such, better devices and methods are needed for reducing fluid egress from the targeted portion of the GI tract to enable improved imaging quality, longer imaging time, and lumen distensibility.

### SUMMARY

There is a need for new and useful devices and methods for at least partially occluding a bodily lumen, for example during an endoscopy procedure. One aspect of the present disclosure is directed to a device configured to block at least a portion of a lumen of a GI tract during an endoscopy procedure. In some embodiments, the device includes an inner shaft having a proximal portion, a distal portion, and defining a lumen therethrough and a first aperture; and an outer shaft having a proximal end and a distal end coupled to the distal portion of the inner shaft, and defining a lumen therethrough. In some embodiments, the outer shaft further includes a flexible member and defines a second aperture positioned proximally on the outer shaft relative to the flexible member. In some embodiments, the inner shaft extends through the lumen of the outer shaft, such that at least a portion of the outer shaft is axially translatable toward and away from the distal portion of the inner shaft. In some embodiments, the flexible member is movable between an unexpanded configuration and an expanded configuration when at least a portion of the outer shaft is translated axially toward or away from the distal portion of the inner shaft.

In any of the preceding embodiments, the flexible member is positioned proximal to the distal end of the outer shaft.

In any of the preceding embodiments, the flexible member is about 0.1 inches to 5 inches from the distal end of the outer shaft.

In any of the preceding embodiments, the flexible member is about 0.8 inches to 1.2 inches from the distal end of the outer shaft.

In any of the preceding embodiments, the inner shaft further includes a stop configured to restrict movement of the outer shaft relative to the inner shaft and thus the flexible member.

In any of the preceding embodiments, the inner shaft further includes an extension indicator on a surface of the inner shaft that indicates an axial translation length required to move the outer shaft to expand the flexible member from the unexpanded configuration to the expanded configuration.

In any of the preceding embodiments, the extension indicator further includes a negative extension indicator indicating a state of over-expansion of the flexible member.

In any of the preceding embodiments, the inner shaft further includes a tip on the distal portion of the inner shaft, such that the distal tip includes a valve configured to prevent liquid from exiting the distal tip of inner shaft.

In any of the preceding embodiments, the device further includes a liquid injection port coupled to the proximal portion of the inner shaft.

In any of the preceding embodiments, the device further includes an infusion device coupled to the liquid injection port configured to deliver the liquid through the liquid injection port, through the first and second apertures, and into the GI tract of the patient proximal to the flexible member.

In any of the preceding embodiments, the flexible member includes a proximal end and a distal end, such that the proximal end of the flexible member is coupled to the outer shaft and the distal end of the flexible member is coupled to the inner shaft.

In any of the preceding embodiments, the device includes a handle having a proximal end coupled to the inner shaft and a distal end coupled to the outer shaft, such that the distal end of the handle is axially translatable to move the proximal end of the outer shaft toward and away from the distal portion of the inner shaft.

In any of the preceding embodiments, at least a first half of the flexible member includes a plurality of struts.

In any of the preceding embodiments, the flexible member further includes a cover configured to encase the plurality of struts.

In any of the preceding embodiments, the plurality of struts encloses a filler material.

In any of the preceding embodiments, the flexible member comprises a plurality of hydratable beads, such that the beads are configured to swell from an unexpanded state to an expanded state.

In any of the preceding embodiments, the plurality of hydratable beads is configured to expand when a liquid is applied through the one or both of the inner shaft and the outer shaft.

In any of the preceding embodiments, the flexible member includes or is formed of a braided material.

In any of the preceding embodiments, the flexible member further includes a cover configured to encase the braided material.

In any of the preceding embodiments, the braided material encloses a filler material.

In any of the preceding embodiments, the braided material comprises Nitinol.

In any of the preceding embodiments, the first aperture is substantially aligned with the second aperture when the flexible member is in the expanded configuration.

In any of the preceding embodiments, the flexible member comprises a balloon.

In any of the preceding embodiments, the flexible member is coated with an expandable material.

In any of the preceding embodiments, the expandable material includes thermoplastic polyurethane.

In any of the preceding embodiments, the inner shaft and outer shaft are substantially rotationally fixed relative to one another.

In any of the preceding embodiments, the flexible member expands to a diameter of substantially 2 cm to 4 cm.

Another aspect of the present disclosure is directed to a device configured to block at least a portion of a lumen of a GI tract during an endoscopy procedure. In some embodiments, the device includes an elongate body having a proximal end and a distal end and defining a lumen therethrough. In some embodiments, the elongate body further includes a flexible member and defines at least two apertures, a first aperture positioned proximally on the elongate body relative to the flexible member and a second aperture being configured to inflate the flexible member. In some embodiments, the flexible member is inflatable to an expanded configuration from an unexpanded configuration when a liquid flows through the lumen of the elongate body and out the second aperture of the elongate body.

In any of the preceding embodiments, the elongate body further defines a second lumen configured to receive a guidewire therethrough.

In any of the preceding embodiments, the first aperture is configured to deliver liquid into the GI tract.

Another aspect of the present disclosure is directed to a method for occluding at least a portion of a lumen of a GI tract during an endoscopy procedure. In some embodiments, the method includes: positioning a distal end of an elongate member adjacent to a proximal side of a stricture in a GI tract of a patient; advancing a flow reducing device through a lumen defined by the elongate member and through the stricture in the GI tract of the patient, such that the flow reducing device includes a flexible member and defines one or more apertures; expanding the flexible member of the flow reducing device from an unexpanded configuration to an expanded configuration distal to the stricture in the GI tract; advancing an ultrasound endoscope (echoendoscope) into the GI tract of the patient; infusing a liquid into the GI tract through the one or more apertures in the flow reducing device, such that flow of the liquid past the flexible member is restricted when the flexible member is in the expanded configuration; and imaging at least a portion of the GI tract of the patient with the echoendoscope.

In any of the preceding embodiments, the elongate member is an endoscope

In any of the preceding embodiments, the method further includes reducing a rate of flow of the liquid around the flexible member and through the downstream GI tract of the patient to less than 230 ml/minute.

In any of the preceding embodiments, the downstream GI tract includes one or more of: esophagus, stomach, a small intestine, a large intestine of the patient.

In any of the preceding embodiments, positioning further includes advancing the elongate member down the GI tract of the patient such that the distal end of the elongate member is positioned adjacent to the proximal side of a stricture.

In any of the preceding embodiments, the method further includes advancing a guidewire through the lumen of the elongate member and through the stricture of the GI tract of the patient, such that the flow reducing device is passed over the guidewire and through the stricture.

In any of the preceding embodiments, the method further includes removing the elongate member from the GI tract before advancing the echoendoscope into the GI tract.

In any of the preceding embodiments, infusing further includes coupling a liquid injection port to a proximal end of the flow reducing device, such that the liquid injection port is configured to deliver the liquid through a lumen defined by the flow reducing device and out the one or more apertures of the flow reducing device into the GI tract.

In any of the preceding embodiments, the GI tract includes one or more of: an esophagus, a stomach, a small intestine, a large intestine.

In any of the preceding embodiments, the method further includes advancing a entero-enterostomy device through a lumen of the echoendoscope.

In any of the preceding embodiments, the method further includes performing a entero-enterostomy procedure.

In any of the preceding embodiments, the method further includes collapsing the flexible member from the expanded configuration to the unexpanded configuration.

In any of the preceding embodiments, the method further includes removing the flow reducing device from the GI tract.

In any of the preceding embodiments, the method further includes attaching a handle to the flow reducing device to facilitate expansion or contraction of the flexible member.

In any of the preceding embodiments, the method further includes actuating the handle to manipulate the outer shaft relative to the inner shaft.

In any of the preceding embodiments, the method further includes removing the echoendoscope from the GI tract.

In any of the preceding embodiments, the flow reducing device further includes: an inner shaft have a proximal portion, a distal portion, and defining a lumen therethrough and a first aperture of the one or more apertures; and an outer shaft having a proximal end and a distal end coupled to the distal portion of the outer shaft, and defining a lumen therethrough. In some embodiments, the outer shaft defines a second aperture of the one or more apertures which is positioned proximally on the outer shaft relative to the flexible member. In some embodiments, the inner shaft extends through the lumen of the outer shaft; such that at least a portion of the outer shaft is axially translatable toward and away from the distal portion of the inner shaft to manipulate the flexible member.

In any of the preceding embodiments, the method further includes contacting an inner surface of a lumen of the GI tract with at least a portion of a perimeter of the flexible member.

Another aspect of the present disclosure is directed to a method for occluding at least a portion of a lumen of a GI tract during an endoscopy procedure. In some embodiments, the method includes: advancing a flow reducing device through a stricture in a GI tract of a patient, such that the flow reducing device comprises a flexible member and defines one or more apertures; expanding the flexible member of the flow reducing device from an unexpanded configuration to an expanded configuration distal to the stricture in the GI tract; advancing an echoendoscope into the GI tract of the patient; infusing a liquid into the GI tract through the one or more apertures in the flow reducing device, such that flow of the liquid past the flexible member is restricted when the flexible member is in the expanded configuration; and imaging at least a portion of the GI tract of the patient with the echoendoscope.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 schematically illustrates an endoscopic ultrasound procedure without the use of the devices and methods described elsewhere herein.

FIG. 2 schematically illustrates advancement of an endoscope through a GI tract of a patient.

FIG. 3 schematically illustrates optional advancement of a guidewire through a lumen of the endoscope of FIG. 2 and past a stricture in a GI tract of a patient.

FIG. 4 schematically illustrates advancement of a flow reducing device, in an unexpanded configuration, through a GI tract of a patient and past a stricture.

FIG. 5 schematically illustrates deployment of a flexible member of the flow reducing device of FIG. 4, from an unexpanded configuration to an expanded configuration, thereby at least partially occluding the GI tract of the patient.

FIG. 6 schematically illustrates removal of the endoscope from the GI tract of the patient.

FIG. 7 schematically illustrates advancement of an echoendoscope through the GI tract of the patient.

FIG. 8 schematically illustrates attachment of a liquid injection port and an infusion device to an inner shaft of the flow reducing device to deliver a liquid into the GI tract of the patient.

FIG. 9 schematically illustrates removal of the liquid injection port and the infusion device of FIG. 8 from the flow reducing device.

FIG. 10 schematically illustrates contraction of the flow reducing device from an expanded configuration to an unexpanded configuration.

FIG. 11 schematically illustrates removal of the flow reducing device from the GI tract of the patient.

FIG. 12 shows one embodiment of a flow reducing device in an unexpanded configuration.

FIG. 13 shows the flow reducing device of FIG. 12 in an expanded configuration.

FIG. 14 shows one embodiment of a flexible member of a flow reducing device, the flexible member including or being formed of a braided material.

FIG. 15 shows one embodiment of the flexible member of FIG. 14 including a cover for at least partially encasing the braided material.

FIG. 16 shows a zoomed in view of the flexible member of FIG. 14 in an expanded configuration.

FIG. 17A shows a zoomed in view of the flexible member of FIG. 14 in an unexpanded configuration.

FIG. 17B shows a zoomed in view of one or more friction locks between an inner shaft and an outer shaft of a flow reducing device.

FIG. 18 shows another embodiment of a flexible member of a flow reducing device, the flexible member being in an unexpanded configuration.

FIG. 19 shows the flexible member of FIG. 18 in an expanded configuration.

FIG. 20 shows a view along line B-B of the flexible member of FIG. 19.

FIG. 21 shows an exploded view of the flow reducing device of FIG. 19.

FIG. 22 shows an exploded view of another embodiment of a flow reducing device, similar to that of FIG. 21.

FIG. 23 shows a zoomed-in view of a flexible member of the flow reducing device of FIG. 22.

FIG. 24 shows another embodiment of a flexible member, in an expanded configuration, of a flow reducing device.

FIG. 25 shows a cross-sectional view of the flexible member of FIG. 24.

FIG. 26 shows one embodiment of a handle configured for use with any of the flow reducing devices described herein.

FIG. 27 shows the handle of FIG. 26 in an actuated state, with a flexible member in an expanded configuration.

FIG. 28 shows manipulation of the handle of FIG. 26 by a user.

FIG. 29A shows cross-sectional view A-A of FIG. 27 of a clamp for securing the handle of FIG. 26 to an inner shaft of a flow reducing device.

FIG. 29B shows a zoomed-in view of the clamp of FIG. 29A.

FIG. 30 shows one embodiment of a handle configured for use with any of the flow reducing devices described herein.

FIG. 31 shows the handle of FIG. 30 in an actuated state, where a flexible member would be in an expanded configuration.

FIG. 32 shows one embodiment of a handle configured for use with any of the flow reducing devices described herein.

FIG. 33 shows the handle of FIG. 32 in an actuated state, where a flexible member would be in an expanded configuration.

FIG. 34 shows one embodiment of a handle configured for use with any of the flow reducing devices described herein.

FIG. 35 shows the handle of FIG. 34 in an actuated state, where a flexible member would be in an expanded configuration.

FIG. 36 shows an isometric view of the handle of FIG. 34.

FIG. 37 shows one embodiment of a handle configured for use with any of the flow reducing devices described herein.

FIG. 38 shows the handle of FIG. 37 in an actuated state, where a flexible member would be in an expanded configuration.

FIG. 39 shows another embodiment of a flexible member, in an unexpanded configuration, of a flow reducing device.

FIG. 40 shows a zoomed-in view of a plurality of hydratable elements of the flexible member of FIG. 39.

FIG. 41 shows a zoomed-in view of a cover of the flexible member of FIG. 39, the cover having a perforated distal surface.

FIG. 42 shows a cross-sectional view of the lumens of the flow reducing device of FIG. 39.

FIG. 43 shows the flow reducing device of FIG. 39 in an expanded configuration.

FIG. 44 shows another embodiment of a flow reducing device.

FIG. 45 shows a zoomed-in view of section E of FIG. 44, which shows a flexible member of the flow reducing device of FIG. 44.

FIG. 46 shows a cross-sectional view of various lumens of the flow reducing device of FIG. 44.

FIG. 47 shows another cross-sectional view of various lumens of the flow reducing device of FIG. 44.

FIG. 48 shows one embodiment of a plug for the handle and/or devices of FIGS. 44-53.

FIG. 49 shows a perspective view of a handle for the flow reducing device of FIGS. 39-47, the handle configured to receive the plug of FIG. 48.

FIG. 50 shows an outer portion of the handle of FIG. 49.

FIG. 51 shows the handle of FIGS. 49-50 in an open configuration, configured to receive a liquid for inflating a flexible member.

FIG. 52 shows the handle of FIGS. 49-50 in a closed configuration.

FIG. 53 shows the handle of FIGS. 49-50 with the plug of FIG. 48 inserted in a lumen of the handle.

FIG. 54 shows a method of occluding at least a portion of a bodily lumen using any of the preceding embodiments of flow reducing devices.

FIG. 55 shows a method of reducing flow through a bodily lumen using a flow reducing device comprising two or more flexible members.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Disclosed herein are devices and methods for at least partially occluding a bodily lumen of a body. Occluding may include, but not be limited to, blocking the bodily lumen; obstructing the bodily lumen; reducing flow of a liquid through the bodily lumen; and capturing or blocking a particle, mass, object, etc. moving through the bodily lumen.

As used herein, a bodily lumen may include, but not be limited to, a GI tract, a mouth, an esophagus, a stomach, a small intestine, a large intestine, a blood vessel, an artery, a vein, a heart chamber, a renal chamber or tract, a kidney, a urinary tract, a bladder, a urethra, a vaginal canal, a uterus, a cervix, a trachea, a bronchial tube, a bronchus, a bronchiole, a respiratory tract, a lymphatic duct, a biliary duct, a pancreatic duct, etc.

The devices and methods described herein and obvious variations thereof may be used in a variety of procedures. Exemplary, non-limiting embodiments of procedures or medical practice areas include: endoscopy, ultrasound imaging, thrombectomy (e.g., capturing clot particles), gastrojejunostomy, biopsy (e.g., capturing tissue samples), imaging, diagnostics, ablation, stenting (e.g., lumen-apposing metal stent), etc.

As will be appreciated by one of skill in the art, the telescoping shaft assembly of any of the embodiments may be used in any other embodiments described herein without significantly departing from the original design. Similarly, any of the single shaft embodiments described herein may be used in any other embodiments described herein without significantly departing from the original design. Further, as will be appreciated by one of skill in the art, any of the flexible members described herein may be mechanically expanded, expanded via liquid infusion, expanded via hydration of one or more elements within the flexible member, or any other method or means of expanding a flexible member. Further, as will be appreciated by one of skill in the art, although terms like expanded and unexpanded are used, any number of intermediate configurations or intermediate expansion or contraction states between extremes is contemplated herein.

In some embodiments, any one or more of the components may be manufactured as or sold as a kit. For example, a kit may include any one or more of: a flow reducing device, an endoscope, an infusion device, a liquid injection port, an echoendoscope, or a combination thereof. A kit may include a flow reducing device with various interchangeable flexible members, such that one flexible member is removable and replaceable with another flexible member.

As described herein, the flexible member of the flow reducing devices may function to reduce a flow rate of liquid through the bodily lumen, dam or obstruct at least a portion of the bodily lumen, prevent liquid flow through the bodily lumen, etc. The flexible members described herein may include or at least partially formed of Nitinol, thermoplastic polymers, thermoset polymers, polyether ether ketone, or like materials known in the art.

As described herein, the flexible member may expand from an unexpanded configuration to an expanded configuration. As used herein, expansion may include movement from a first diameter to a second diameter, the first diameter being less than the second diameter. Alternatively, or additionally, expansion may include increasing a volume of the flexible member from a first volume to a second volume, the first volume being less than the second volume. Volume includes one or both of: the volume of the space enclosed by the flexible member and the volume of the actual cover or material of the flexible member. Volume includes but is not limited to: a volume of space enclosed by the expanded member and a volume of the material (e.g., flexible member, cover, etc.) that expands or contracts with or without filling, respectively. Alternatively, or additionally, expansion may include increasing a surface area in contact with at least part of a bodily lumen, for example a GI tract. The flexible member may be expanded and contracted once each during a procedure or more than once to achieve proper placement of the device during a procedure.

As used herein, a liquid, inflation liquid, filling liquid, etc. may include water, saline, contrast, drugs (e.g., anti-coagulants, thrombolytics, etc.), etc.

As used herein, fluid may include water, saline, contrast, drugs (e.g., anti-coagulants, thrombolytics, etc.), gases, air, etc.

The flow reducing devices described herein may have an exchange length of greater than about 1.5×, greater than about 2×, greater than about 2.5×, greater than about 3×, greater than about 3.5×, etc. of the endoscope working channel. For example, the exchange length of a flow reducing device may be greater than about 3.5 m, greater than about 4 m, greater than about 4.5 m, greater than about 5 m, etc.

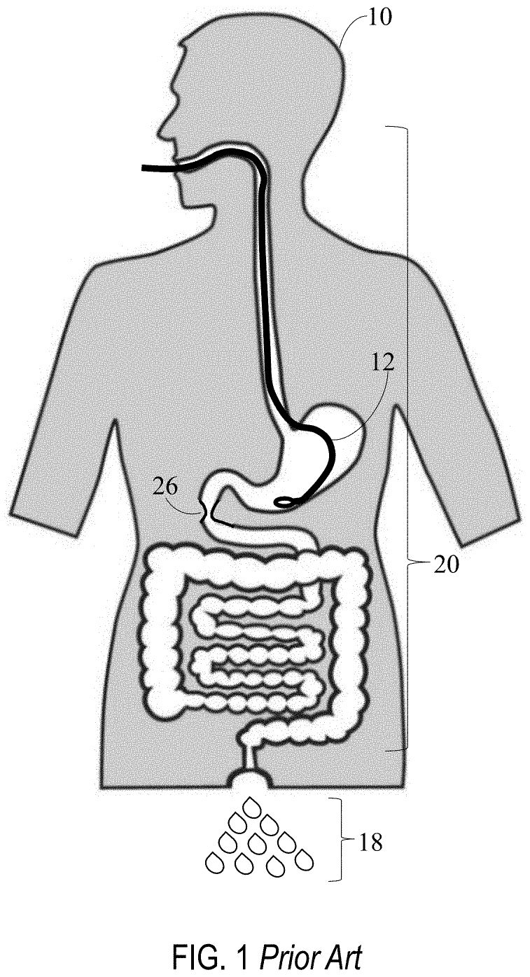

FIG. 1 schematically illustrates an endoscopic ultrasound procedure without the use of the devices and methods described elsewhere herein. Liquid is infused into a targeted portion of the GI tract 20 of the patient 10 using any method known in the art (e.g., catheter, endoscope, etc.). Since there is nothing to restrict downstream flow, the liquid flow rate 18 through the GI tract is high. One of skill in the art will appreciate that while liquid flow rate 18 is shown at the exit of the GI tract, the liquid flow rate 18 may be more localized in the GI tract 20 and be passed through the GI tract over a period time after the procedure. After the targeted portion of GI tract 20 is sufficiently filled with liquid, an echoendoscope 12 is advanced into the GI tract 20 to image the targeted portion of the GI tract 20, for example distal from a stricture 26 in the GI tract 20. The quality of the ultrasound imaging is limited by the time during which the GI tract 20 is filled with liquid, which is directly proportional to the liquid flow rate 18 through the GI tract 20. The devices and methods described herein sought to solve this liquid flow rate 18 problem with a technical solution directed toward reducing the liquid flow rate 18 through the GI tract 20 to allow more time for better imaging, more extensive investigation of the tissues in the GI tract 20, to present a bigger target for fine needle aspiration, etc. or really any bodily lumen to which the devices and methods are applicable.

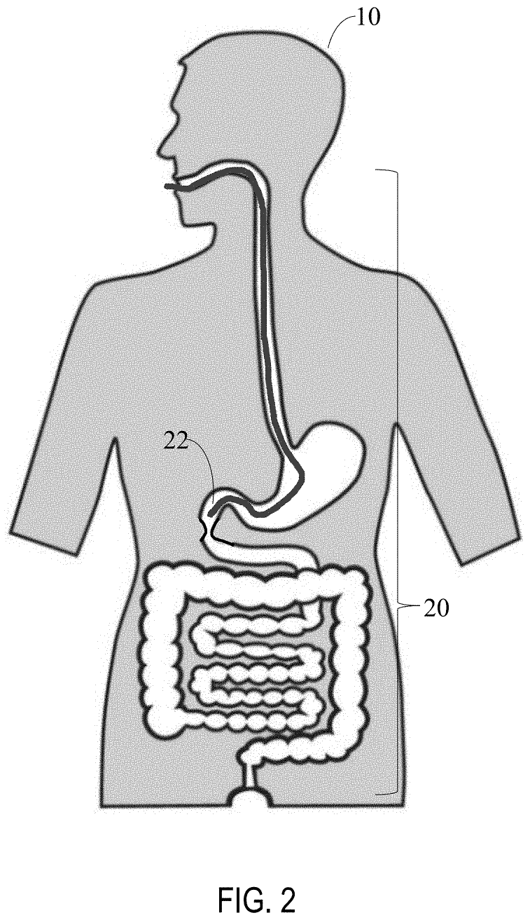

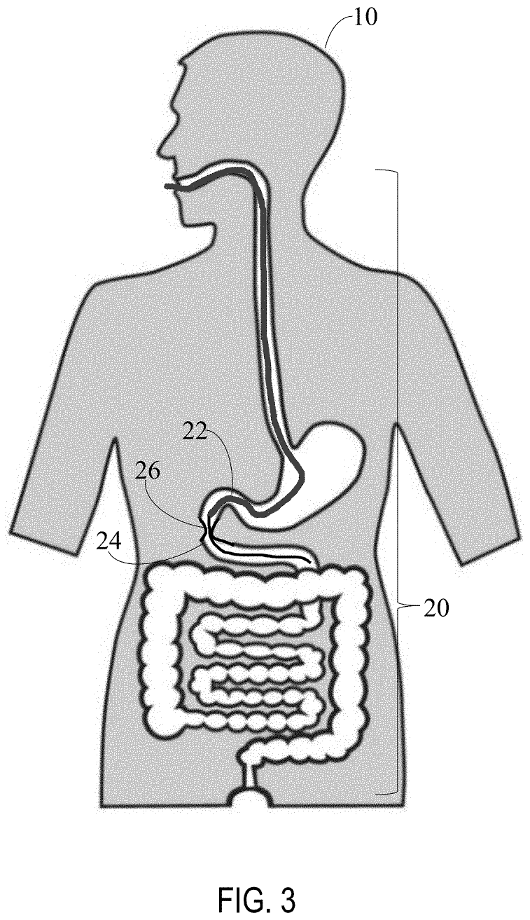

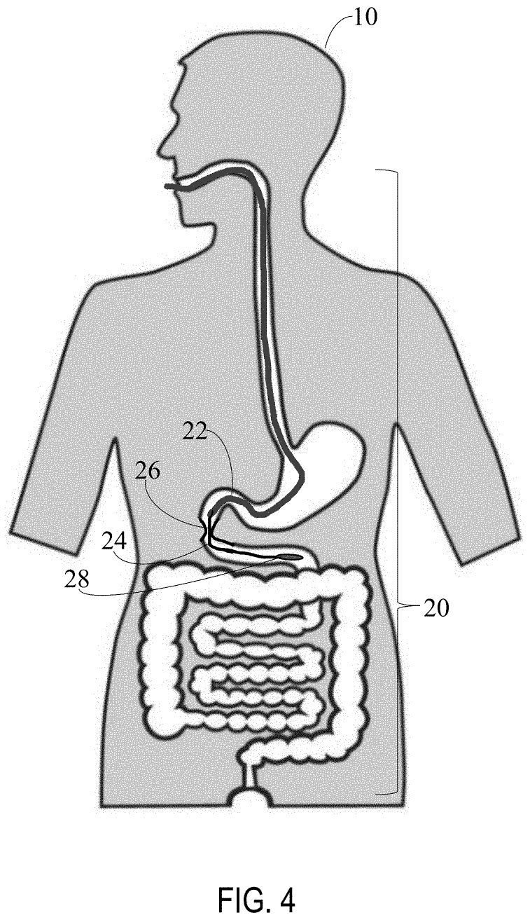

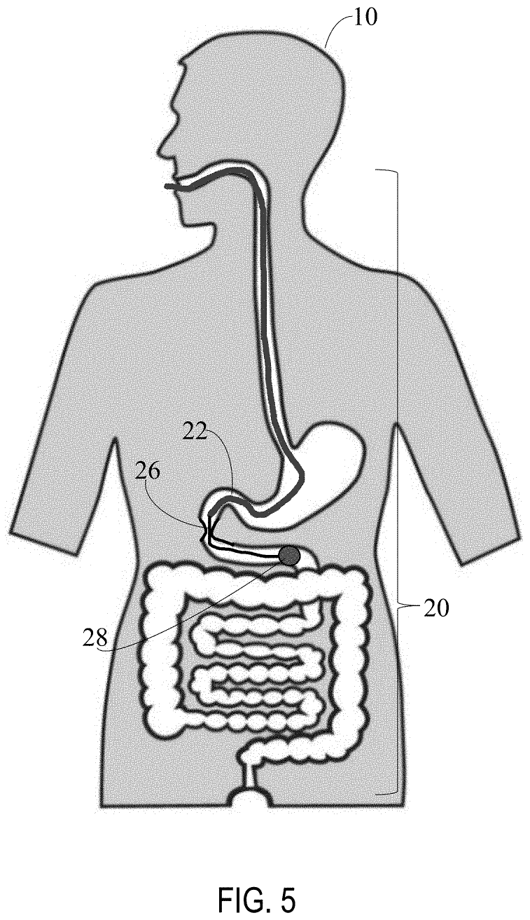

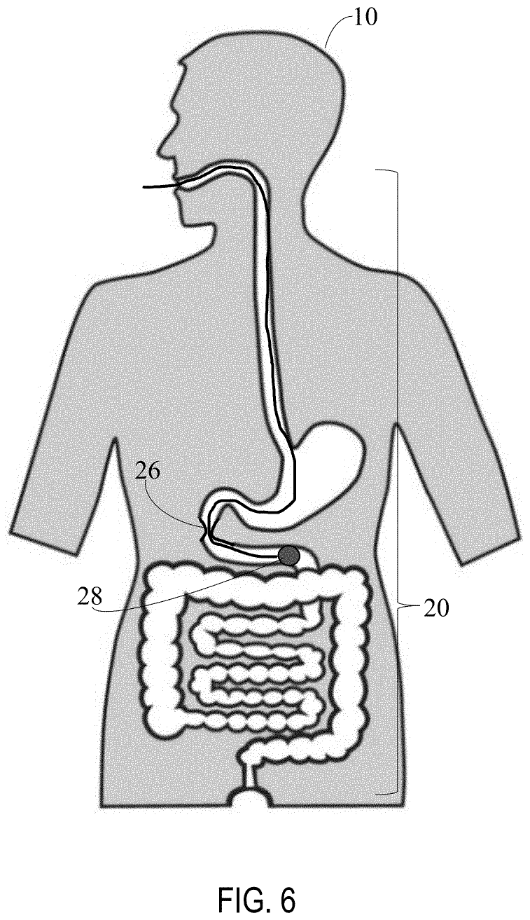

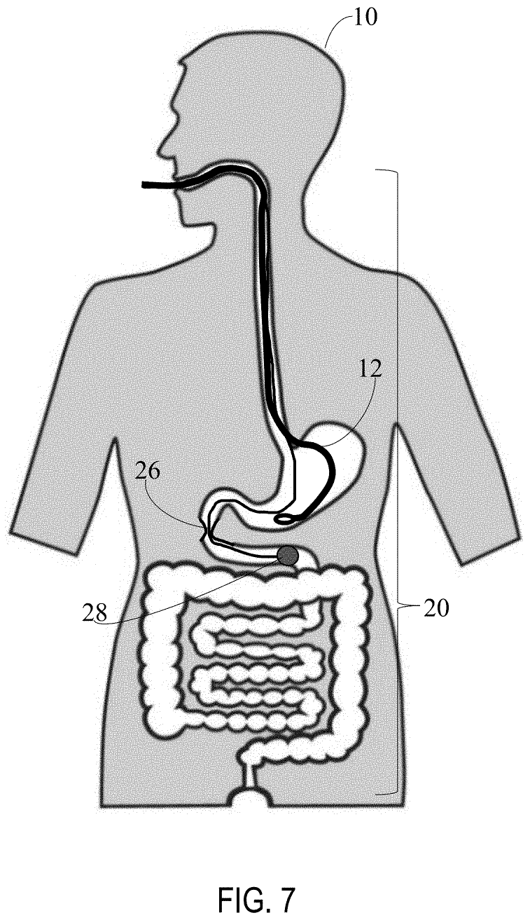

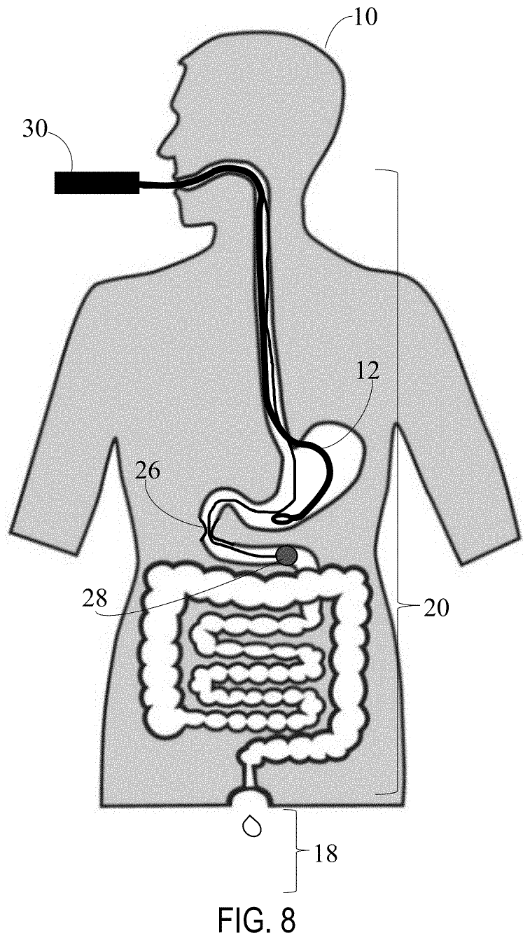

FIGS. 2-11 illustrate an exemplary method of the embodiments and technical solutions described elsewhere herein. FIG. 2 schematically illustrates advancement of an endoscope 22 through a GI tract 20 of a patient 10. Optionally, as shown in FIG. 3, a guidewire 24 is advanced through a working channel of the endoscope 22 and past a stricture 26 in a GI tract 20 of a patient 10. As shown in FIG. 4, a flow reducing device 28, in an unexpanded configuration, is advanced over the guidewire 24 or just through a lumen of the endoscope 22 through a GI tract 20 of a patient 10 and past a stricture 26. The guidewire 24 is then removed and the flexible member of the flow reducing device 28 is deployed from an unexpanded configuration to an expanded configuration, as shown in FIG. 5, to thereby at least partially occlude the GI tract 20 of the patient 10. The endoscope 22 is then removed from the GI tract 20 of the patient, as shown in FIG. 6. An echoendoscope 12 is advanced through the GI tract 20 of the patient 10, as shown in FIG. 7. A handle 30 (e.g., comprising a liquid injection port (e.g., tuohy borst, female luer port, etc.) and an infusion device (e.g., infusion pump, syringe, etc.) is then attached to a proximal portion (e.g., inner shaft or elongate body) of the flow reducing device to deliver a liquid into the GI tract 20 of the patient 10, as shown in FIG. 8. The liquid that is infused largely remains proximal to the flow reducing device 28 to provide an enhanced imaging environment for the echoendoscope 12 that is not constrained by the timeline associated with liquid exiting the GI tract 20, since the flow reducing device 28 reduces or prevents liquid from exiting the GI tract 20, as shown by liquid 18 exiting the GI tract. The handle 30 is removed from the proximal end of the flow reducing device 28, as shown in FIG. 9, and the flow reducing device 28 is moved from an expanded configuration to an unexpanded configuration, as shown in FIG. 10. In embodiments where a gastro-enterostomy is performed, location X indicates where the small intestine (distal duodenum/proximal jejunum) would be connected to the stomach. The flow reducing device 28 is then removed from the GI tract 20 of the patient 10, as shown in FIG. 11. In some embodiments, the flow reducing device 28 is removed with the echoendoscope 12; in other embodiments, the flow reducing device 28 and the echoendoscope 12 are removed sequentially, in either order (e.g., flow reducing device then echoendoscope or echoendoscope then flow reducing device).

In some embodiments, as shown in FIG. 55, a flow reducing device includes two or more flexible members such that a liquid infused into the bodily lumen is at least partially retained between the flexible members. The two or more flexible members may be expanded sequentially, simultaneously, or substantially simultaneously and contracted sequentially, simultaneously, or substantially simultaneously.

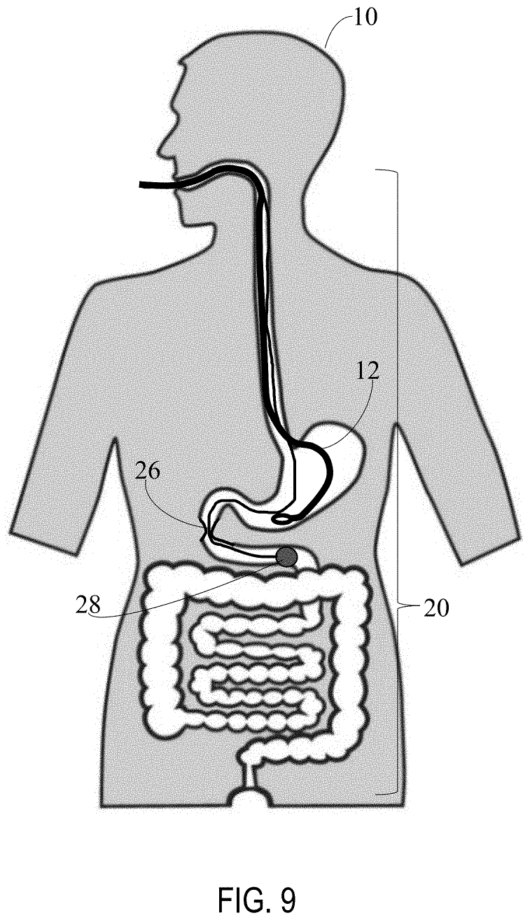

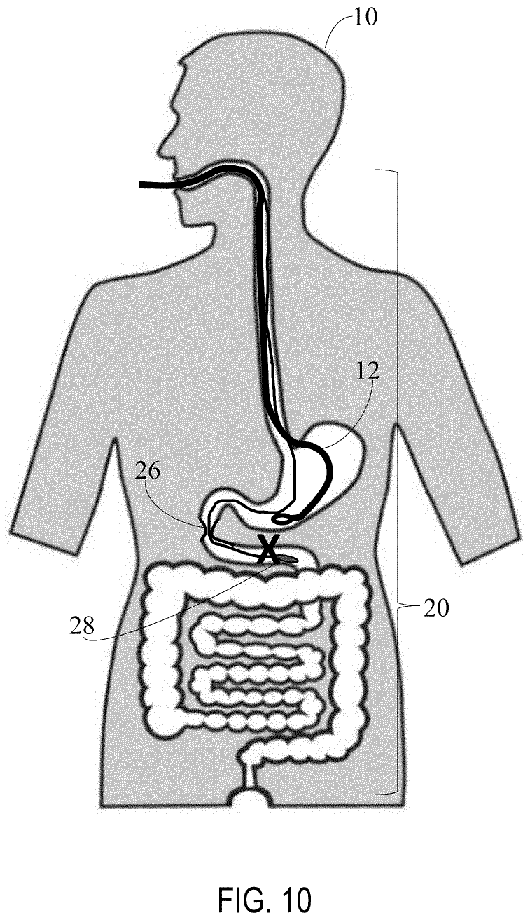

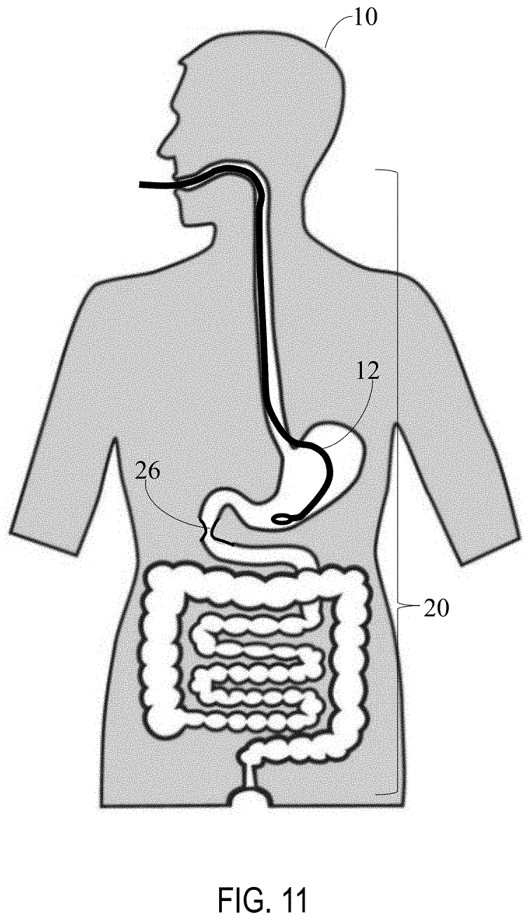

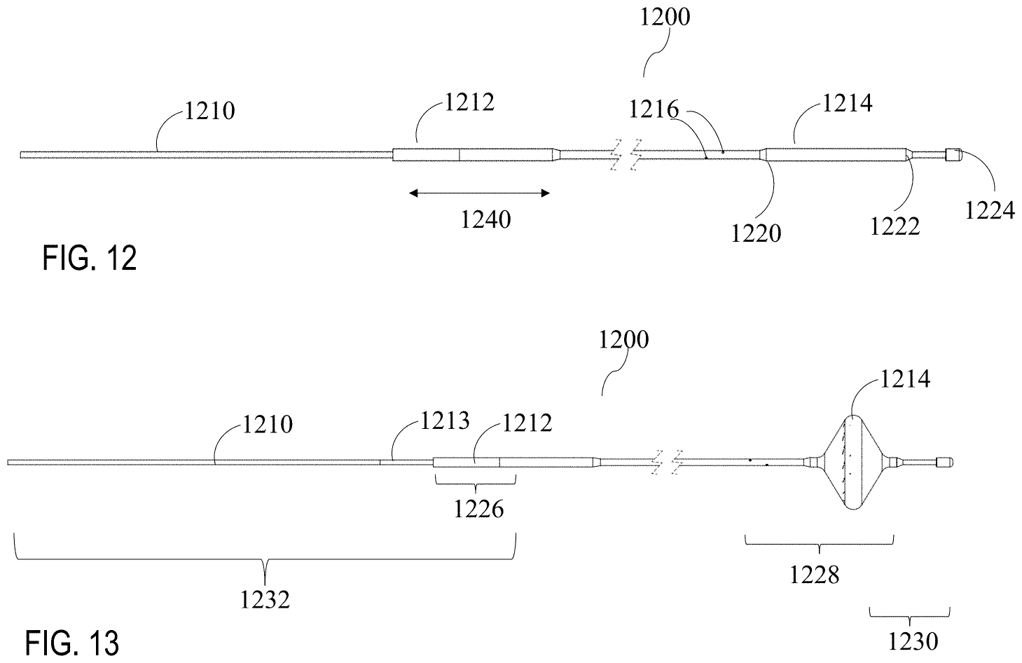

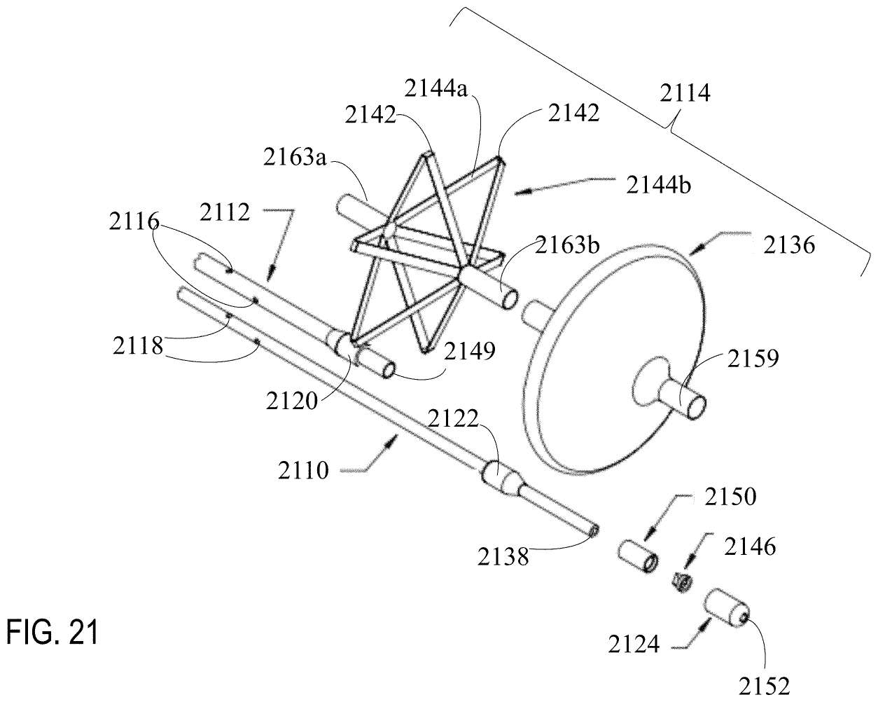

FIGS. 12-13 show one embodiment of a flow reducing device 1200 in an unexpanded and expanded configuration, respectively. A device configured to block at least a portion of a bodily lumen includes an inner shaft 1210 having a proximal portion 1232, a distal portion 1230, and defining a lumen 2138 (shown, e.g., in FIG. 21) therethrough and a first aperture 2118 (shown, e.g., in FIG. 21). The lumen 2138 of the inner shaft 1210 is configured to receive a guidewire therethrough, such that the flow reducing device 1200 may be passed over a guidewire to reach a target or desired position in a bodily lumen. The device 1200 further includes an outer shaft 1212 having a proximal end 1226 and a distal end 1228 coupled to the distal portion 1230 of the inner shaft 1210, and defining a lumen 2149 (shown, e.g., in FIG. 21) therethrough. The outer shaft 1212 further includes a flexible member 1214 and defines a second aperture 1216 positioned proximally on the outer shaft 1212 relative to the flexible member 1214. The inner shaft 1210 extends through the lumen of the outer shaft 1212, such that the inner shaft 1210 and the outer shaft 1212 form at least a partially telescoping assembly or the inner shaft 1210 and the outer shaft 1212 form concentric shafts or tubes. For example, at least a portion of the outer shaft 1212 is axially translatable 1240 toward and away from the distal portion 1230 of the inner shaft 1210 to expand the flexible member 1214 (outer shaft 1212 moves towards distal portion 1230 of inner shaft 1210) and contract the flexible member 1214 (outer shaft 1212 moves away from distal portion 1230 of inner shaft 1210). In some embodiments, the inner shaft 1210 and outer shaft 1212 are substantially rotationally fixed relative to one another. In some such embodiments, the first aperture 1216 is substantially aligned with the second aperture 2118 when the flexible member 1214 is in the expanded configuration. In other embodiments, the inner shaft 1210 and outer shaft 1212 are rotatable relative to one another, such that the flexible member 1214 is torquable in a clockwise or counterclockwise direction. In some embodiments, a torqued configuration of the flexible member 1214 in an expanded configuration imparts differential liquid flow or movement characteristics to the liquid that is proximal to the flexible member 1214 and into the GI tract.

The flexible member 1214 is positioned proximal to the distal end, shown as cap 1224, of the inner shaft 1210. In some embodiments, the flexible member 1214 is about 0.1 inches to about 5 inches from the distal end of the inner shaft 1210. In some embodiments, the flexible member 1214 is about 0.8 inches to about 1.2 inches from the distal end, shown as cap 1224, of the inner shaft 1210.

The inner shaft 1210, optionally, includes an extension indicator 1213, for example a positive extension indicator indicates an axial translation length required to move the outer shaft 1212 to expand the flexible member 1214 from the unexpanded configuration to the expanded configuration, for example to prevent hyper or hypo extension of the flexible member 1214. In some embodiments, extension indicator 1213 further includes a negative extension indicator indicating a state of over-expansion of the flexible member 1214. For example, the flexible member expands to a diameter of substantially or about 2 cm to about 4 cm, about 1.5 cm to about 4.5 cm, about 3 cm to about 5 cm, about 1 cm to about 3 cm, etc. In other embodiments, a proximal end 1226 of the outer shaft 1212 defines a window or cutout such that an extension indicator on the inner shaft 1210 is visible through the outer shaft 1212. The extension indicator 1213 may include one or more of: a color, a visual pattern, a tactile pattern (e.g., nodules, ribbing, etc.), haptics, etc. For example, the positive extension indicator may be a green color and the negative extension indicator may be a red color. Any of the flow reducing devices described herein may optionally include an extension indicator.

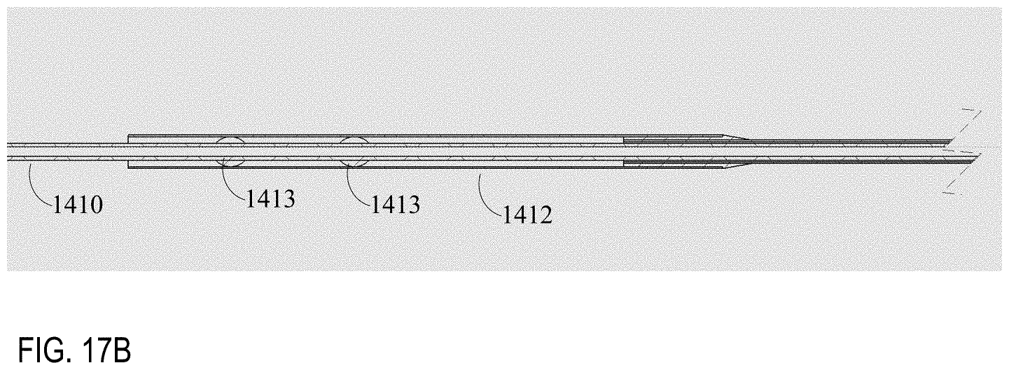

Alternatively, or additionally, a tactile indicator, for example a mechanical stop, may exist between the inner shaft 1210 and the outer shaft 1212, such that outer shaft 1212 is prevented from sliding past the inner shaft 1210 at a certain length to prevent hyper or hypo extension of the flexible member 1214. One or more friction locks 1413, as shown in FIG. 17B, may be positioned on an outer diameter of the inner shaft 1410 and/or on an inner diameter of the outer shaft 1412 to prevent the inner shaft 1410 from sliding past the outer shaft 1412 when the flexible member is in the expanded configuration. The friction locks prevent slippage or sliding between the outer shaft 1412 and the inner shaft 1410 by creating friction between the outer diameter of the inner shaft 1410 and the inner diameter of the outer shaft 1412. Such friction may prevent inadvertent changing of the expanded state of the flexible member 1214. Any of the flow reducing devices described herein may optionally include one or more mechanical stops or locks.

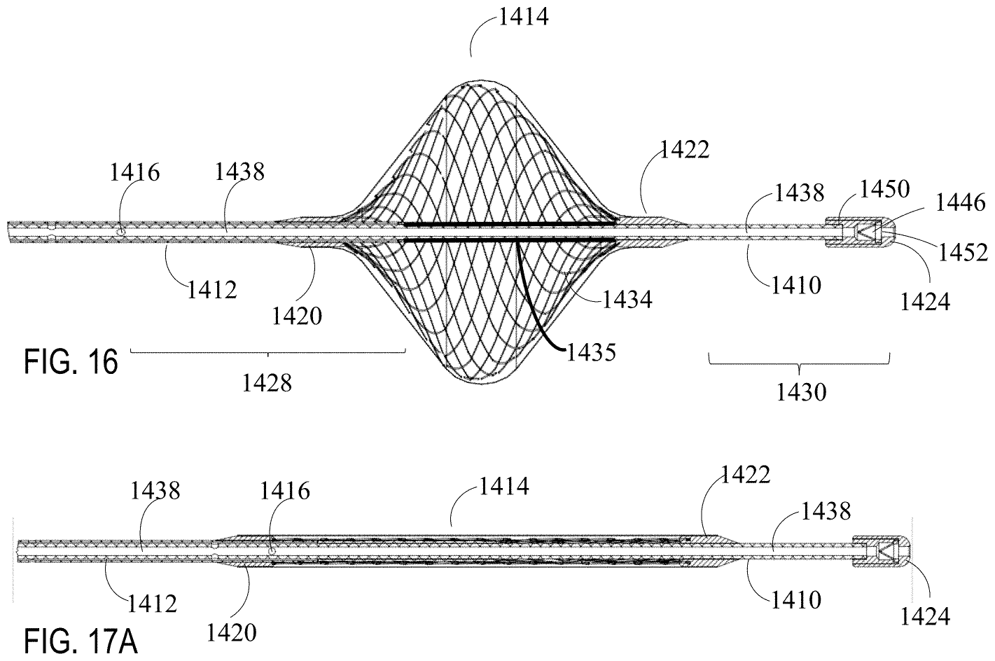

Further, any of the flow reducing devices described herein may include an expansion stop 1435, as shown in FIG. 16. Expansion stop 1435 includes one or more of the following: a movable or fixed concentric tube on the inner shaft 1410 that a distal end 1428 of the outer shaft 1412 butts up against when expanding the flexible member 1414; and/or one or more fixed protrusions, rings, or the like on an outer diameter of the inner shaft 1410 that the outer shaft 1412 butts up against when expanding the flexible member 1414. Alternatively, a distal end 1428 of the outer shaft 1412 may extend beyond coupling 1420, such that when the flexible member 1414 expands, the distal end 1428 of the outer shaft 1412 butts up against coupling 1422 between the inner shaft 1410 and the flexible member 1410. In one embodiment, as shown in FIG. 16, the distal end 1428 of the outer shaft 1412 pushes up against expansion stop 1435 when moving from the unexpanded configuration to the expanded configuration of the flexible member 1414. The expansion stop 1435 prevents the outer shaft 1412 from over or hyper expanding the flexible member 1414 during deployment. If the flexible member 1414 is over or hyper expanded, the flexible member 1414 may start to flatten, thereby creating a thin rim that would be irritating or even harmful to the bodily lumen in which it is positioned. The flattened shape may also, or alternatively, fold over rendering its liquid flow reducing or block capacity reduced or less effective.

Further, as shown in FIGS. 12-13, the flexible member includes a proximal end and a distal end, such that the proximal end 1220 of the flexible member 1214 is coupled to the outer shaft 1212 and the distal end 1222 of the flexible member 1214 is coupled to the inner shaft 1210. The flexible member 1214 is coupled to the outer shaft 1212 at position 1220, and the flexible member 1214 is coupled to the inner shaft 1210 at position 1222. The coupling between the flexible member 1214 and the outer shaft 1212 and the flexible member 1214 and the inner shaft 1210 may be via glue, adhesive, soldering, welding, brazing, mechanical linkage (e.g., keyed or complementary surfaces), solvent bonding, or any other method known to one of skill in the art.

The inner shaft 1210 further includes a distal tip cover 1224 that includes a valve (e.g., duckbill valve) therein, as shown and described elsewhere herein, that prevents liquid that is transported through the lumen of the inner shaft 1210 from exiting the distal end or tip of the flow reducing device 1200 while allowing a guidewire therethrough.

In some embodiments, a system for at least partially occluding a bodily lumen includes a flow reducing device 1200 and additionally, a liquid injection port (e.g., tuohy borst valve, female luer port, etc.) coupled to the proximal portion 1232 of the inner shaft 1210 and an infusion device (e.g., pump, syringe, etc.) coupled to the liquid injection port configured to deliver the liquid through the liquid injection port, through the first and second apertures 1216, 2118, and into the GI tract of the patient proximal to the flexible member 1214. Apertures 1216, 2118 may have a diameter of about 0.005 to about 0.05 inches, for example about 0.01 to about 0.05 inches.

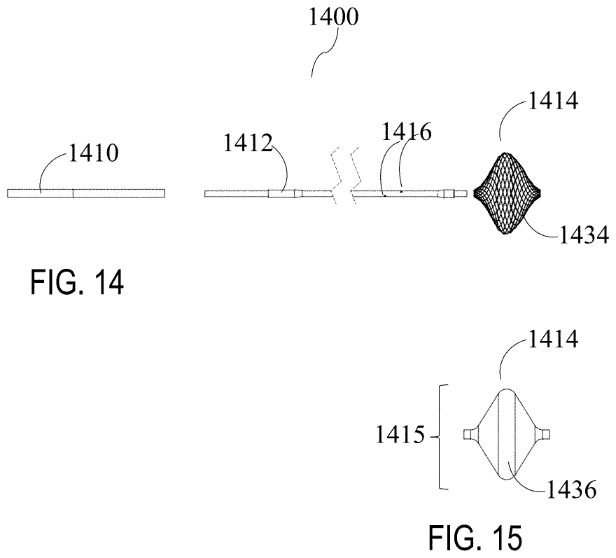

FIGS. 14-17A show another embodiment of a flow reducing device 1400. Flexible member 1414 of flow reducing device 1400 includes or is at least partially formed of an alternative material 1434, for example braided Nitinol, stainless steel, cobalt-chrome alloy, titanium, gold, platinum, silver, iridium, tantalum, tungsten, etc. In some embodiments, the braided material further encloses a filler material or hydratable material, as described elsewhere herein. Alternatively, or additionally, the alternative material 1434 of the flexible member 1414 may also be formed of or comprise a tube that is cut in a pattern or a bundle of fibers substantially axially aligned that would expand when compressed axially (similar to a stent). The flexible member 1414 may be further covered by a cover 1436, for example including or formed at least in part of a thermoplastic polymer, a thermoset polymer, or similar material. The cover 1436 may at least partially or wholly encase the flexible member 1414. For example, in some embodiments, the cover 1436 may only cover a proximal side or portion 1415 of the flexible member 1414. Flow reducing device 1400 further includes an inner shaft 1410, outer shaft 1412, apertures 1416, inner shaft lumen 1428, flexible member to outer shaft coupling 1420, and flexible member to inner shaft coupling 1422, as described above in connection with FIGS. 12-13. Additional detail of the distal tip of the inner shaft 1210 is shown in FIGS. 16-17A. The distal end 1430 of the inner shaft 1210 includes distal tip 1450, valve 1446, and cap 1424 secured to the distal tip 1450. Cap 1424, in some embodiments, defines an aperture 1452 therethrough such that a guidewire or other elongate device can be passed through the lumen 1438 of the inner shaft 1210 and out the aperture 1452 defined by cap 1424. Valve 1446 and cap 1424 prevent liquid that is transported through lumen 1438 from being expelled out of a distal end of the flow reducing device 1400, while still allowing a guidewire or other elongate device to pass through cap 1424 through aperture 1452. The flexible member 1414 of FIGS. 16-17A is moveable between an expanded configuration (FIG. 16) and an unexpanded configuration (FIG. 17A), similar to that described above in connection with FIGS. 12-13.

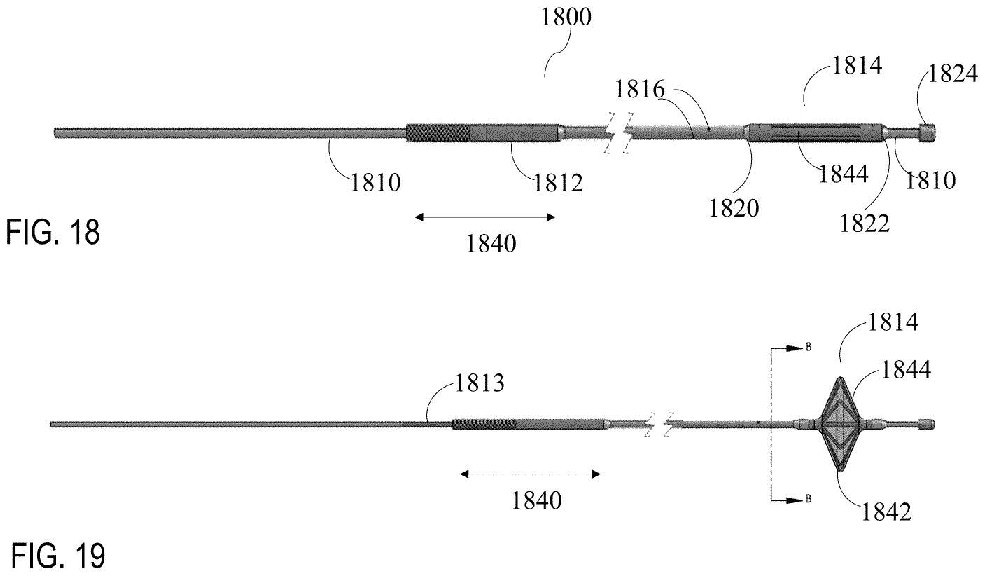

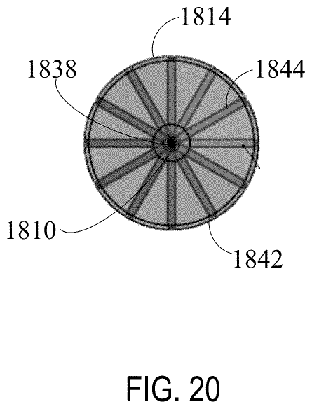

FIGS. 18-21 show another embodiment of a flexible member 1814 of a flow reducing device 1800. The flexible member 1814 is movable between an unexpanded configuration, as shown in FIG. 18, and an expanded configuration, as shown in FIGS. 19-21. Similar to embodiments described above, the flow reducing device of FIGS. 18-21 includes an inner shaft 1810, 2110 (defining lumen 1838, 2138 therethrough) and an outer shaft 1812, 2112 (defining lumen 2149 therethrough) that are axially translatable 1840 relative to one another; a flexible member 1814, 2114; one or more apertures 2116 in outer shaft 2112; one or more apertures 2118 in inner shaft 2110; a first or proximal coupling 1820, 2120 between the outer shaft 1812, 2112 and the flexible member 1814, 2114; a second or distal coupling 1822, 2122 between the inner shaft 1810, 2110 and the flexible member 1814, 2114; and a distal cap 1824, 2124 with similar internal components (e.g., valve 2146, guidewire lumen 2152, distal tip 2150 of inner shaft 2110), as described above. However, in this embodiment, at least a first half of the flexible member 1814, 2114 includes a plurality of struts 1844, 2144. As will be appreciated, any number of struts is contemplated: 2, 3, 4, 5, 6, 7, 8, 9, 10, or 12 struts, for example. The plurality of struts 1844, 2144 includes a plurality of joints 1842, 2142. FIG. 21 shows an exploded view of the flow reducing device 1800 of FIG. 18. For example, each strut may be formed of a proximal strut 2144a and a distal strut 2144b coupled together at joint 2142 and a proximal hub 2163a and distal hub 2163b. Joint 2142 enables the flexible member 2114 to move from an unexpanded configuration in which the proximal strut 2144a is substantially or about 180 degrees relative to the distal strut 2144b about joint 2142 to an expanded configuration in which the proximal strut 2144a is substantially or about 20 to about 70 degrees relative to the distal strut 2144b about joint 2142. The flexible member 2114 may further include a cover 2136, coupled to inner shaft 2110 via distal cover hub 2159, the cover being configured to encase the plurality of struts 2144. Optionally, the plurality of struts 2144 encloses a filler material in some embodiments.

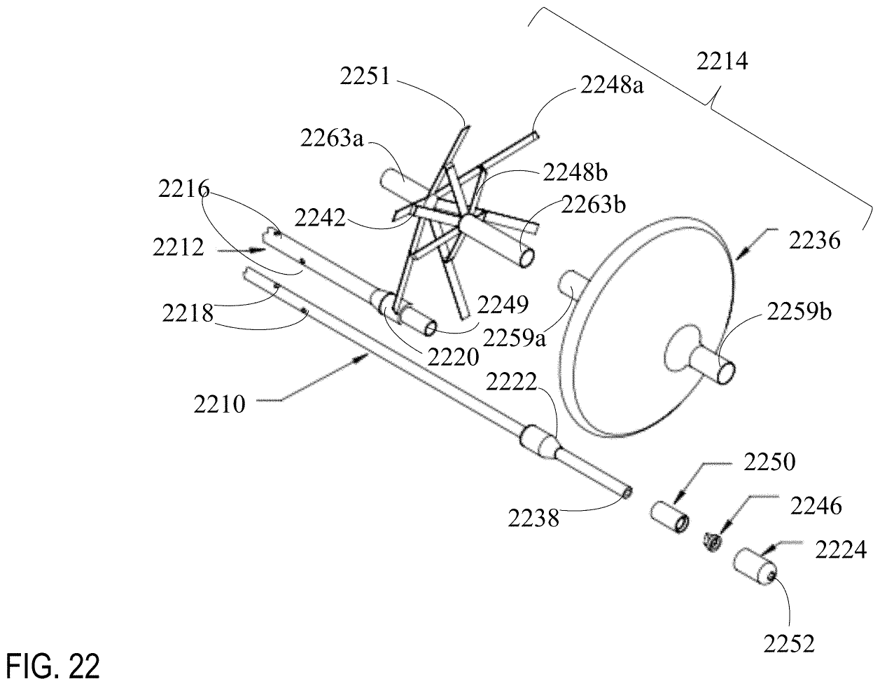

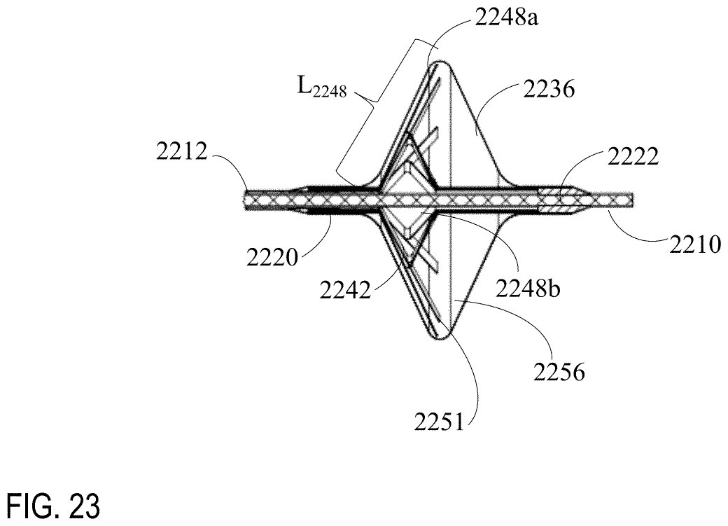

FIGS. 22-23 show another embodiment of a flexible member 2214 of a flow reducing device, the flexible member being similar to that of FIGS. 18-21. As shown in FIG. 22, the flow reducing device includes similar components as the flow reducing devices described above: an inner shaft 2210 (defining lumen 2238 therethrough) and an outer shaft 2212 (defining lumen 2249 therethrough) that are axially translatable relative to one another; a flexible member 2214; one or more apertures 2216 in outer shaft 2212; one or more apertures 2218 in inner shaft 2210; a first or proximal coupling 2220 between the outer shaft 2212 and the flexible member 2214; a second or distal coupling 2222 between the inner shaft 2210 and the flexible member 2214; and a distal cap 2224 with similar internal components (e.g., valve 2246, guidewire lumen 2252, distal tip 2250 of inner shaft 2210), as described above. However, in this embodiment, at least a first half of the flexible member 2214 includes a plurality of struts 2248. The plurality of struts 2248 includes a plurality of joints 2242. For example, each strut may be formed of a proximal strut 2248a and a distal strut 2248b coupled together at joint 2242 and a proximal hub 2263a and distal hub 2263b. As shown in FIG. 22, the proximal strut 2248a is about twice as long as the distal strut 2248b (2:1 proximal strut:distal strut), such that the joint 2242 is about half-way down the length L₂₂₄₈ of the proximal strut 2248a. In other embodiments, the length ratio between the proximal strut 2248a and the distal strut 2248b is about 1.5:1; about 3:1, about 2.5:1, about 4:1, etc. A free end 2251 of proximal strut 2248a, as shown in FIGS. 22-23, may be rendered atraumatic using post-processing methods or be covered in an atraumatic material in addition to, or alternatively to, cover 2236. Joint 2242 enables the flexible member 2214 to move from an unexpanded configuration in which the proximal strut 2148a is substantially or about 180 degrees relative to the distal strut 2248b about joint 2242 to an expanded configuration in which the proximal strut 2248a is substantially or about 20 to about 70 degrees relative to the distal strut 2248b about joint 2242. The flexible member 2214 may further include a cover 2236 configured to encase the plurality of struts 2248, as shown in FIG. 23. Cover 2236 is coupled to inner shaft 2210 via distal cover hub 2259b and outer shaft 2212 via proximal cover hub 2259a. Cover 2236 may define space or enclosure 2256. Optionally, enclosure 2256 may be fillable with a fluid (e.g., gas, water, drug, etc.) or a filler material, in some embodiments.

In some embodiments of FIG. 23, a concavity of the plurality of struts 2248 may be facing a proximal end of the flow reducing device and a cover 2236 on the plurality of struts 2248 may fit more closely to the struts such that the plurality of struts behave more like a basket for collecting, for example, biopsy samples, clots, etc.

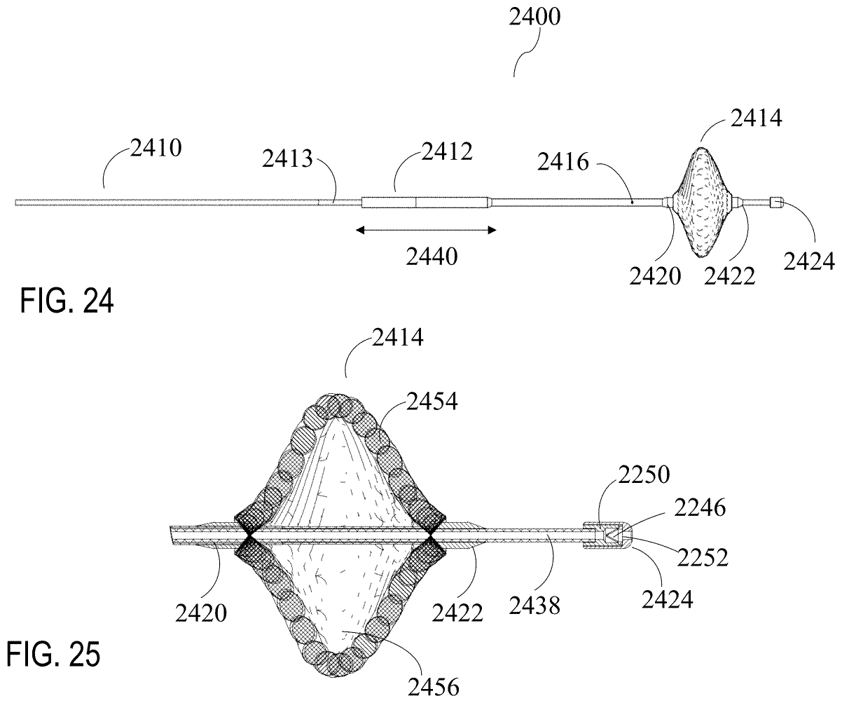

FIGS. 24-25 show another embodiment of a flow reducing device 2400 including flexible member 2414. Flow reducing device 2400 includes similar features as those described above: an inner shaft 2410 and an outer shaft 2412 that are axially translatable 2440 relative to one another; a flexible member 2414; one or more apertures 2416 in outer shaft 2412; a first or proximal coupling 2420 between the outer shaft 2412 and the flexible member 2414; a second or distal coupling 2422 between the inner shaft 2410 and the flexible member 2414; and a distal cap 2424 with similar internal components (e.g., valve, guidewire lumen, distal tip of inner shaft, etc.), as described above. However, in this embodiment, the flexible member 2414 includes or is formed of an alternative material (e.g., braided, laser cut, etc.) that is coated 2454. The braided, coated 2454 flexible member 2414 defines enclosure 2456, as described above. As such, flexible member 2414 does not include a cover per say, but rather a coating 2454 on the alternative material that functions to prevent liquid flow through the flexible member 2414 once it is infused into the GI tract through one or more apertures 2416.

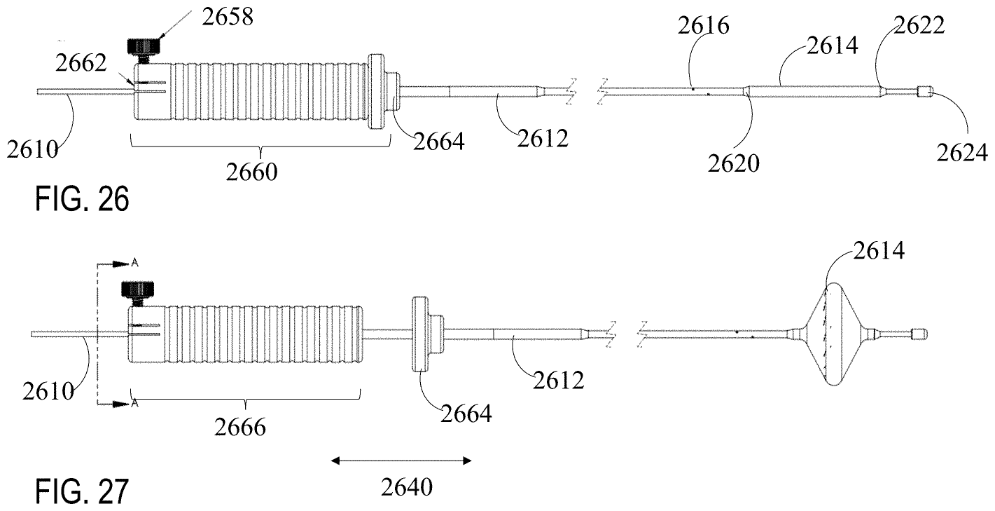

Now turning to FIGS. 26-38, which show various handle configurations for the flow reducing devices described herein. As used herein, handle describes any device that is configured to manipulate a flow reducing device, actuate (e.g., mechanically, fluidly, etc.) a flexible member from an unexpanded state to an expanded state, and as needed, back to an unexpanded state. The handle may be used for actuation, fluid or liquid transport into the flexible member, plugging one or more ports of the flow reducing device, etc.

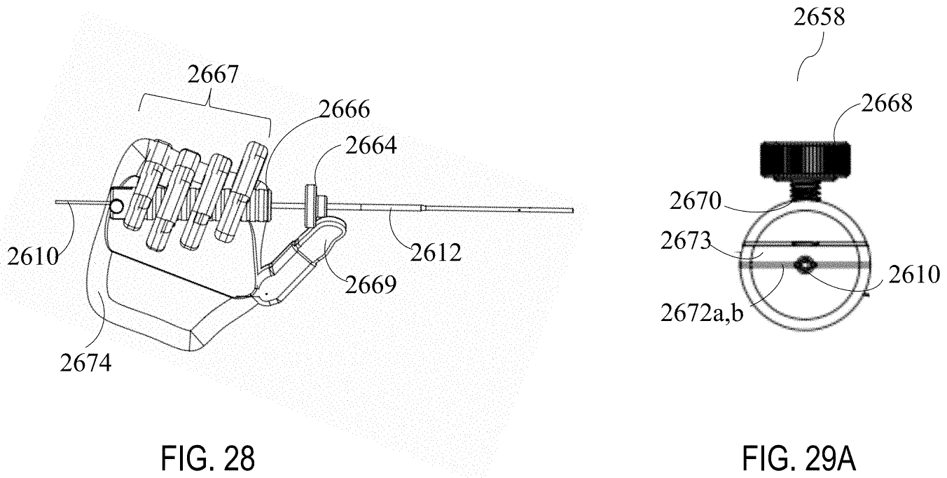

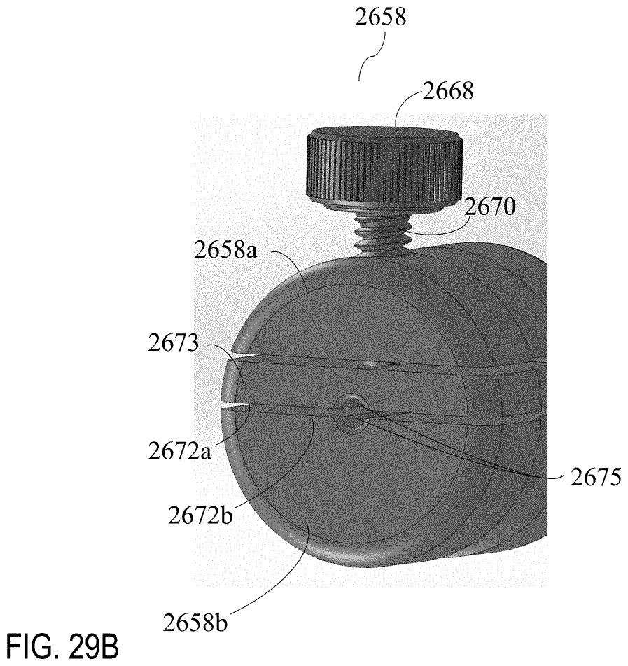

FIGS. 26-29B show one embodiment of a handle 2660 configured for use with any of the flow reducing devices described herein. FIG. 26 shows handle 2660 in an unactuated state, with flexible member 2614 in an unexpanded configuration, and FIG. 27 shows handle 2660 in an actuated state, with flexible member 2614 in an expanded configuration. As shown in FIGS. 26-27, the flow reducing device includes an inner shaft 2610, an outer shaft 2612, a first or proximal coupling 2620 between the outer shaft 2612 and the flexible member 2614; a second or distal coupling 2622 between the inner shaft 2610 and the flexible member 2614; and a distal cap 2624 with similar internal components (e.g., valve, guidewire lumen, distal tip of inner shaft, etc.), as described above. The proximal end 2662 of handle 2660 is coupled to inner shaft 2610 and distal end 2664 of handle 2660 is coupled to outer shaft 2612. As shown in FIG. 28, during use, a user's hand 2674 may grasp handle body 2666 with fingers 2667 and palm and manipulate the distal end 2664 of handle 2660 with thumb 2669. Axial translation 2640 of the outer shaft 2612 relative to the inner shaft 2610 is facilitated by axial translation of distal end 2664 of handle 2660 relative to handle body 2666, such that when distal end 2664 of handle 2660 is moved distally away from handle body 2666, the flexible member 2614 expands into the expanded configuration (FIG. 27) and when the distal end 2664 of handle 2660 is moved proximally toward handle body 2666, the expanded flexible member 2614 moves into the unexpanded configuration (FIG. 26). The proximal end 2662 of handle 2660 is secured to the inner shaft 2610 via clamp 2658. A detailed version of clamp 2658 is shown in FIGS. 29A-29B. Similar clamps to clamp 2658 are used in several embodiments described elsewhere herein.

As shown in FIGS. 29A-29B, clamp 2658 includes an upper clamp body 2658a and a lower clamp body 2658b with movable wedge 2673 therebetween. Actuation (e.g., rotation) of knob 2668 and thereby actuation of screw 2670 moves movable wedge 2673 towards the lower clamp body 2658b to apply force to an inner shaft 2610 secured between an upper clamp surface 2672a and a lower or bottom clamp surface 2672b. The upper clamp surface 2672a and the lower clamp surface 2672b may each include complementary grooves 2675 sized and shaped to receive and secure an inner shaft 2610 therebetween.

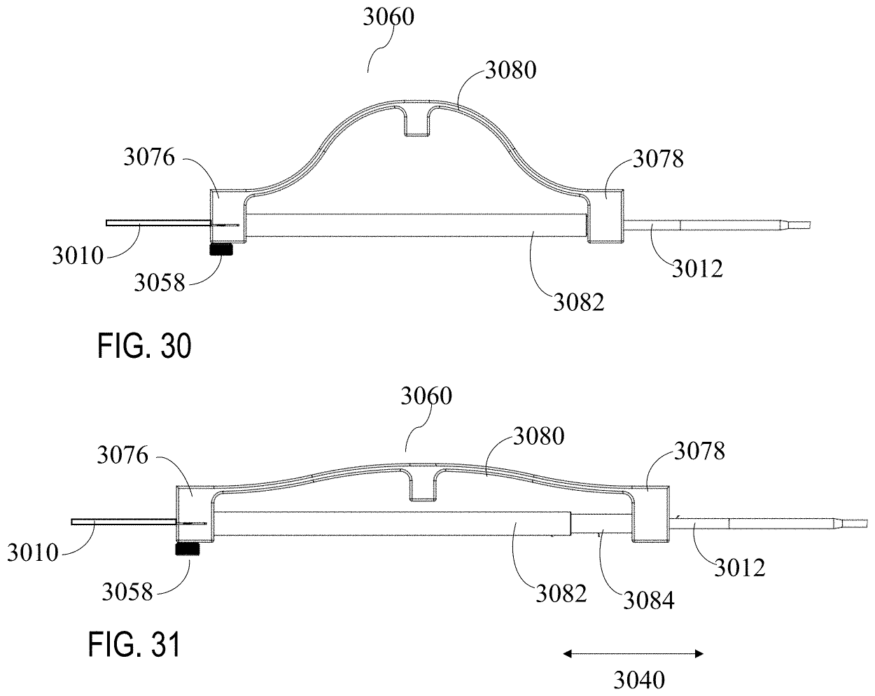

FIGS. 30-31 show another embodiment of a handle 3060 configured for use with any of the flow reducing devices described elsewhere herein. A first end or proximal end 3076 of handle 3060 is coupled to inner shaft 3010 and a second end or distal end 3078 of handle 3060 is coupled to outer shaft 3012. The proximal end 3076 and distal end 3078 of handle 3060 are coupled together via flexible handlebar 3080 and via a first telescoping tube or proximal tube 3082 coupled to first or proximal end 3076 and a second telescoping tube or distal tube 3084 coupled to second or distal end 3078, the distal tube 3084 being axially translatable within a lumen of the proximal tube 3082. Handle 3060 is actuated by squeezing the flexible handlebar 3080 with respect to (i.e., towards) telescoping tubes 3082, 3084. As such, flexible handle bar 3080 flattens and elongates, thereby displacing the distal end 3078 away from the proximal end 3076 or axially translating 3040 the distal end 3078 of handle 3060 relative to the proximal end 3076 of handle 3060 to move a flexible member from an unexpanded configuration (FIG. 30) to an expanded configuration (FIG. 31). For example, as the distal end 3078 of handle 3060 is moved distally, as shown in FIG. 31 (and flexible handlebar 3080 is elongated), distal tube 3084 extends out of a lumen of proximal tube 3082 and the flexible member is expanded. Conversely, as the distal end 3078 of handle 3060 is moved proximally, as shown in FIG. 30 (and flexible handlebar 3080 is not elongated), distal tube 3084 is substantially or fully within a lumen of the proximal tube 3082 and the flexible member is in an unexpanded configuration. Inner shaft 3010 is secured in handle 3060 via clamp 3058, similar to clamp 2658 described above and inner shaft 3010 is coaxially positioned in distal tube 3084, which is coaxially positioned in proximal tube 3082.

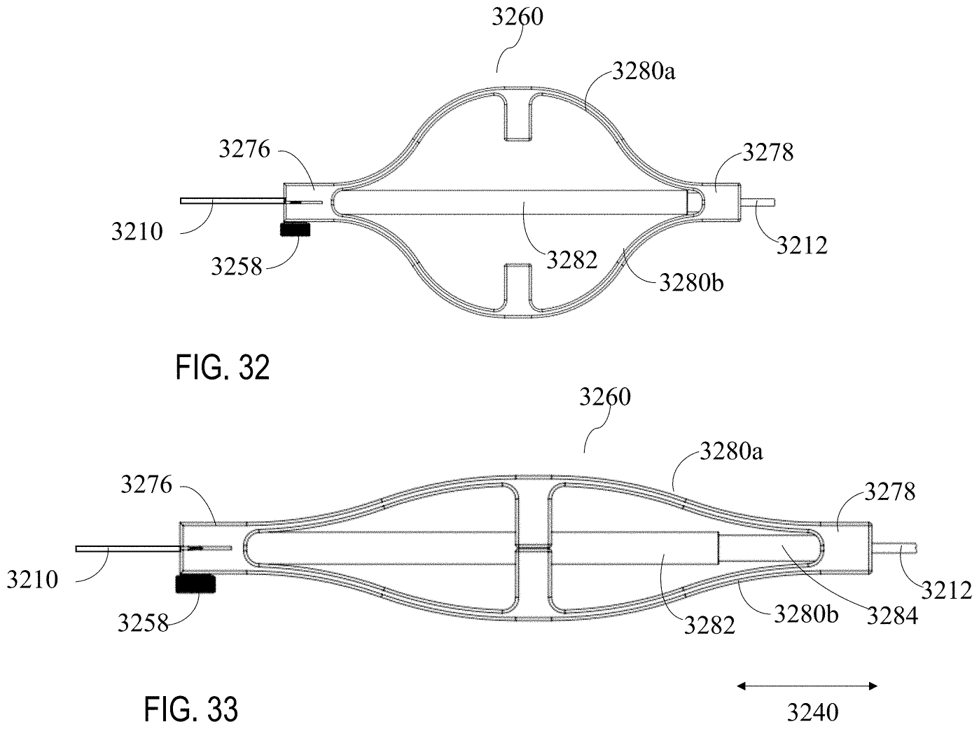

FIGS. 32-33 show another embodiment of a handle 3260 configured for use with any of the flow reducing devices described herein. Handle 3260 is similar to handle 3060 described above including the following features: a first end or proximal end 3276 of handle 3260 coupled to inner shaft 3210; a second end or distal end 3278 of handle 3260 coupled to outer shaft 3212; a proximal end 3276 and distal end 3278 of handle 3260 coupled together via an upper flexible handlebar 3280a and a lower flexible handlebar 3280b; and a proximal end 3276 and distal end 3278 of handle 3260 coupled together via a proximal tube 3282 coupled to the proximal end 3276 and a distal tube 3284 coupled to the distal end 3278. Handle 3260 is actuated by squeezing the upper flexible handlebar 3280a and the lower flexible handlebar 3280b together or towards one another. As such, the upper and lower flexible handlebars 3280a, 3280b flatten and elongate, thereby displacing the distal end 3278 away from the proximal end 3276 or axially translating 3240 the distal end 3278 of handle 3260 relative to the proximal end 3276 of handle 3260 to move a flexible member from an unexpanded configuration (FIG. 32) to an expanded configuration (FIG. 33). For example, as the distal end 3278 of handle 3260 is moved distally, as shown in FIG. 32 (and flexible handlebars 3280a, 3280b are elongated), distal tube 3284 extends out of a lumen of proximal tube 3282 and the flexible member is expanded. Conversely, as the distal end 3278 of handle 3260 is moved proximally, as shown in FIG. 32 (and flexible handlebars 3280a, 3280b are not elongated), distal tube 3284 is substantially or fully within a lumen of the proximal tube 3282 and the flexible member is in an unexpanded configuration. Inner shaft 3210 is secured in handle 3260 via clamp 3258, similar to clamp 2658 described above, and inner shaft 3210 is coaxially positioned in distal tube 3284, which is coaxially positioned in proximal tube 3282.

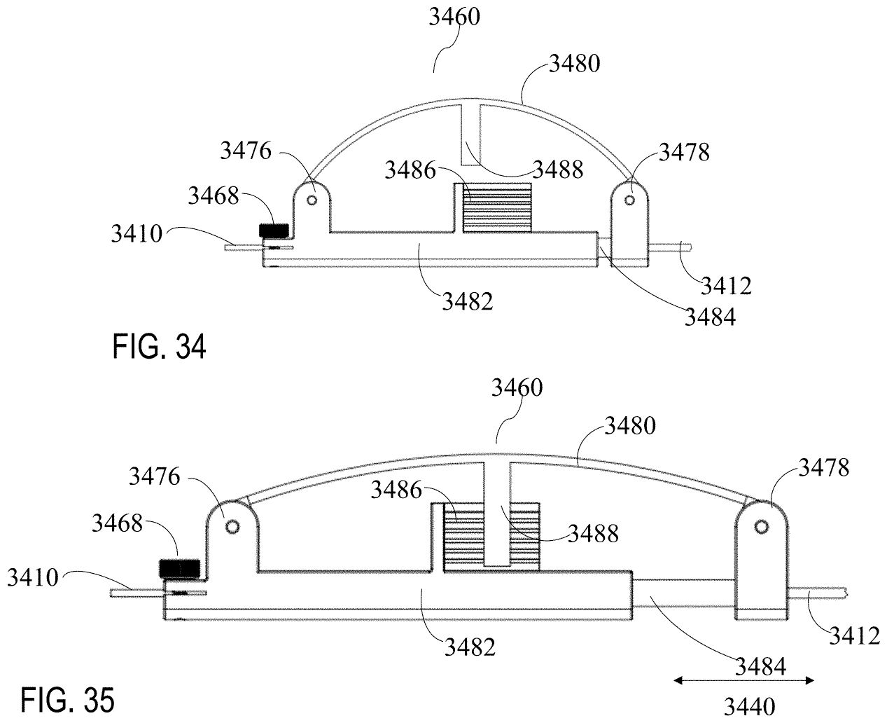

FIGS. 34-36 show another embodiment of a handle 3460 configured for use with any of the flow reducing devices described herein. Handle 3460 has the same general structure as the handles shown in FIGS. 30-33 with a few identifiable differences.

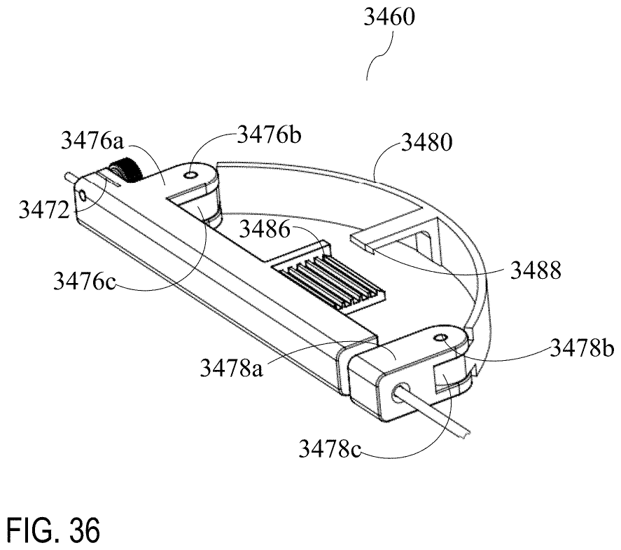

Regarding similarities to FIGS. 30-33, handle 3460 includes: a first or proximal end 3476 of handle 3460 coupled to inner shaft 3410; a second or distal end 3478 of handle 3460 coupled to outer shaft 3412; a proximal end 3476 and distal end 3478 of handle 3460 coupled together via a flexible handlebar 3480; a proximal tube 3482 coupled to the proximal end 3476; and a distal tube 3484 coupled to the distal end 3478. Handle 3460 is actuated by squeezing the flexible handlebar 3480 with respect to (i.e., towards) telescoping tubes 3482, 3484. As such, flexible handle bar 3480 flattens and elongates, thereby displacing the distal end 3478 away from the proximal end 3476 or axially translating 3440 the distal end 3478 of handle 3460 relative to the proximal end 3476 of handle 3460 to move a flexible member from an unexpanded configuration (FIG. 34, FIG. 36) to an expanded configuration (FIG. 35). For example, as the distal end 3478 of handle 3460 is moved distally, as shown in FIG. 35 (and flexible handlebar 3480 is elongated), distal tube 3484 extends out of a lumen of proximal tube 3482 and the flexible member is expanded. Conversely, as the distal end 3478 of handle 3460 is moved proximally, as shown in FIG. 34 (and flexible handlebar 3480 is not elongated), distal tube 3484 is substantially or fully within a lumen of the proximal tube 3482 and the flexible member is in an unexpanded configuration. Inner shaft 3410 is secured in handle 3460 via clamp 3458, similar to clamp 2658 described above, and inner shaft 3410 is coaxially positioned in distal tube 3484, which is coaxially positioned in proximal tube 3482.

Regarding differences from FIGS. 30-33, as best shown in FIG. 36, latch 3488 extends from flexible handlebar 3480 to interact with and couple to stepped extension 3486, so that the handle may secure the flow reducing device into a deployed configuration (flexible member in expanded configuration). Further, proximal end 3476 includes bracket or fork 3476a that is shaped to receive eye 3476c therein, the eye 3476c being secured in fork 3476a via pin 3476b. Distal end 3478 includes a similar configuration with bracket or fork 3478a shaped to receive eye 3478c therein, which is secured therein by pin 3478b. As such, when the flexible handlebar 3480 is actuated (i.e., squeezed towards telescoping tubes 3482, 3484) or returned to an unactuated state, the distal end 3478 moves away from and towards, respectively, proximal end 3476, such that eyes 3476c, 3478c pivot in forks 3476a, 3478a about pins 3476b, 3478b.

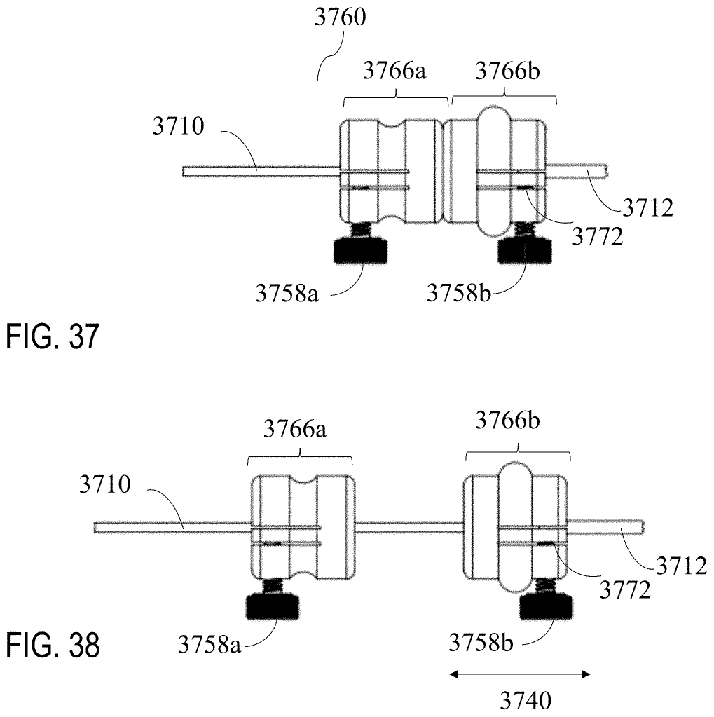

FIGS. 37-38 show another embodiment of a handle 3760 configured for use with any of the flow reducing devices described herein. Handle 3760 uses sets of clamps, similar to that described in FIGS. 29A-29B, to manipulate an inner shaft 3710 relative to an outer shaft 3712. Proximal end body 3766a of handle 3760 is coupled to inner shaft 3710 and distal end body 3766b is coupled to outer shaft 3712, using the mechanisms described in connection with FIGS. 29A-29B. For example, proximal clamp 3758a is coupled to inner shaft 3710 and distal clamp 3758b is coupled to outer shaft 3712. Axial translation 3740 of the distal body 3766b coupled to the outer shaft 3712 towards a distal end of a flow reducing device expands a flexible member of the flow reducing device, while axial translation 3740 of the distal body coupled to the outer shaft towards the proximal end (and thus proximal body 3766a) moves an expanded flexible member into an unexpanded configuration.

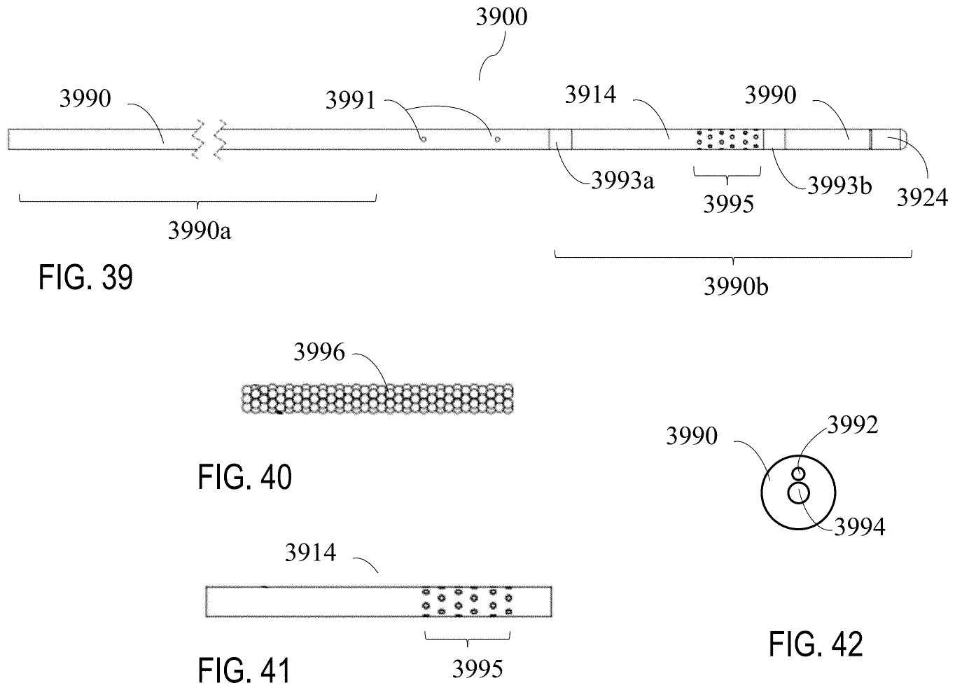

FIGS. 39-47 show various embodiments of flow reducing devices that may include any or all of the features (e.g., extension indicator, mechanical stop, expansion stop, materials, etc.) of any of the other flow reducing devices described elsewhere herein. The embodiments shown in FIGS. 39-47 can exist in multiple configurations. For example, the flow reducing devices may include concentric tubes (inner and outer shaft, as described above and elsewhere herein) or one elongate member, as will be described in further detail below. Further, the flow reducing devices of FIGS. 39-47 may include two or more lumens. For example, in one embodiment, a flow reducing device includes three lumens: one lumen for a guidewire to pass therethrough; one lumen configured to receive inflation liquid therethrough for inflating a flexible member of the flow reducing device; and one lumen configured to receive a liquid for filling a bodily lumen for a procedure. In another embodiment, a flow reducing device includes two lumens: one lumen for a guidewire to pass therethrough, and one lumen configured to receive a liquid for expanding the flexible member and filling a bodily lumen for a procedure.

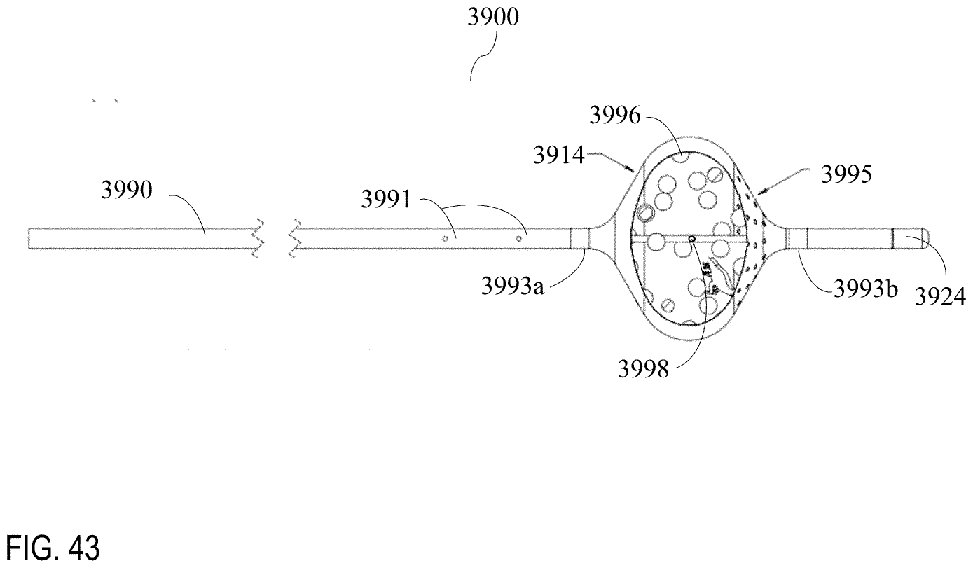

Turning now to FIGS. 39-43, which show an embodiment of a flow reducing device 3900. A device 3900 configured to block at least a portion of a bodily lumen during a procedure includes an elongate body 3990 having a proximal end 3990a and a distal end 3990b and defining a lumen therethrough 3992. The elongate body 3990 further includes a flexible member 3914 that is coupled to the elongate body 3990 at a first or proximal position 3993a and a second or distal position 3993b. The elongate body 3990 defines at least two apertures 3991, 3998. A first aperture 3991 is positioned proximally on the elongate body 3990 relative to the flexible member 3914 and is configured to deliver liquid into a bodily lumen in which the flow reducing device 3900 is positioned. A second aperture 3998 resides within the flexible member 3914 and is configured to inflate the flexible member 3914 with inflation fluid. The flexible member 3914 is inflatable to an expanded configuration, as shown in FIG. 43, from an unexpanded configuration, as shown in FIG. 39, when a liquid flows through the lumen 3992 of the elongate body 3990 and out the second aperture 3998 of the elongate body 3990. In some embodiments, the elongate body 3990 further defines a second lumen 3994 configured to receive a guidewire therethrough, as shown in FIG. 42. Optionally, in some embodiments, the elongate body 3990 further defines a third lumen configured to receive another liquid therethrough, for example to fill a bodily lumen of a patient and/or to fill a flexible member 3914 of a flow reducing device 3900. Elongate body 3990 includes distal cap 3924, similar to that described elsewhere herein and including similar components (e.g., valve, distal tip of elongate body, guidewire lumen), that functions to allow a guidewire to pass therethrough but prevents liquid from escaping from a distal end 3990b of the flow reducing device 3900.

As shown in FIGS. 39-43, flexible member 3914 includes or is formed of an expandable material including a plurality of hydratable beads 3996, such that the beads 3996 are swellable from an unexpanded state to an expanded state when liquid infused into the flexible member 3914 via aperture 3998. The plurality of hydratable beads 3996, when in a hydrated state, may substantially or fully consume the space defined by the flexible member 3914. In other embodiments of the flow reducing device 3900 where there is not just one elongate body but inner and outer shafts, as described elsewhere herein, the plurality of hydratable beads 3996 is configured to expand when a liquid is applied through one or both of the inner shaft and the outer shaft. The flexible member 3914 may further define one or more perforations 3995 on a distal side of the flexible member 3914 for releasing excess liquid from the flexible member 3914 when the plurality of hydratable beads 3996 have reached maximum capacity, a threshold, or equilibrium. In some embodiments, instead of a single elongate member, a flow reducing device 3900 includes an inner shaft and outer shaft configuration as described elsewhere herein.

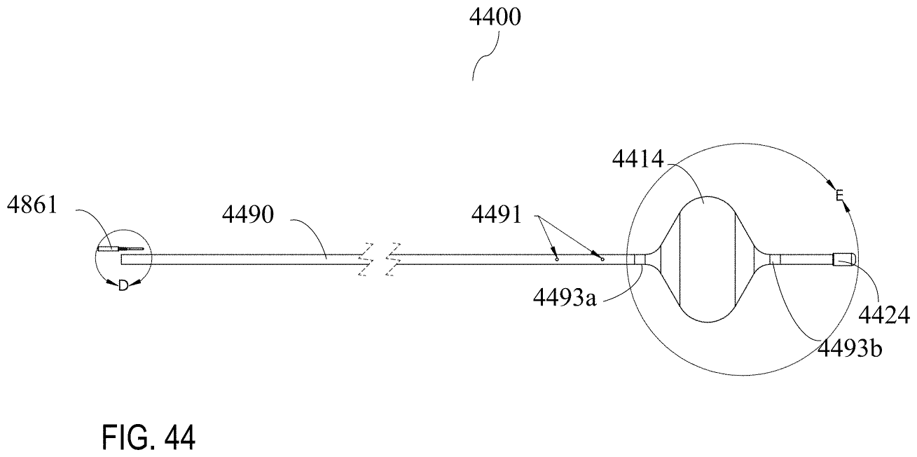

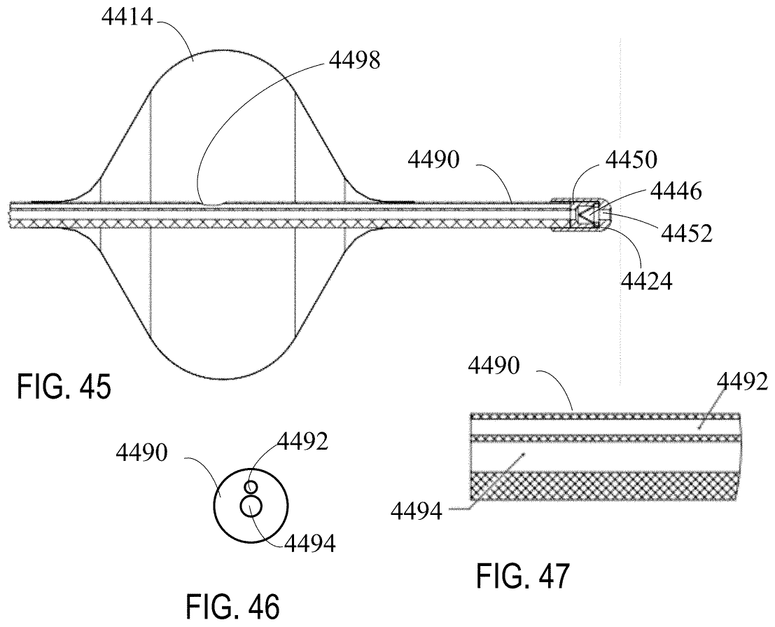

FIGS. 44-47 show another embodiment of a flow reducing device 4400. Flow reducing device 4400 is similar to that described above for 3900 in that includes an elongate body 4490 defining a guidewire lumen 4494 and an infusion lumen 4492 (and optionally a third lumen as described elsewhere herein). The elongate body 4490 further defines a first aperture 4491 and a second aperture 4498. The first aperture 4491 is positioned proximally on the flow reducing device relative to the flexible member 4414 and functions to fill a bodily lumen in which the flow reducing device is positioned. The second aperture 4498 is inside the flexible member 4414 and functions to inflate the flexible member 4414 with a liquid. The flexible member 4414 is coupled to the elongate body 4490 at a proximal coupling position 4493a and a distal coupling position 4493b. The elongate body 4490 further includes a distal cap 4424 that includes a valve 4446, distal tip 4450 of the elongate body 4490, and guidewire lumen 4452, as shown in FIG. 45. As shown in FIGS. 44-47, the flexible member 4414 includes or is formed of an expandable or elastomeric material, for example a balloon, a thermoplastic polyurethane, a thermoset polyurethane, silicone, a polyether ether ketone, an inelastic material (e.g., fills or expands without stretching), etc. In some embodiments, instead of a single elongate member, a flow reducing device 4400 includes an inner shaft and outer shaft configuration as described elsewhere herein.

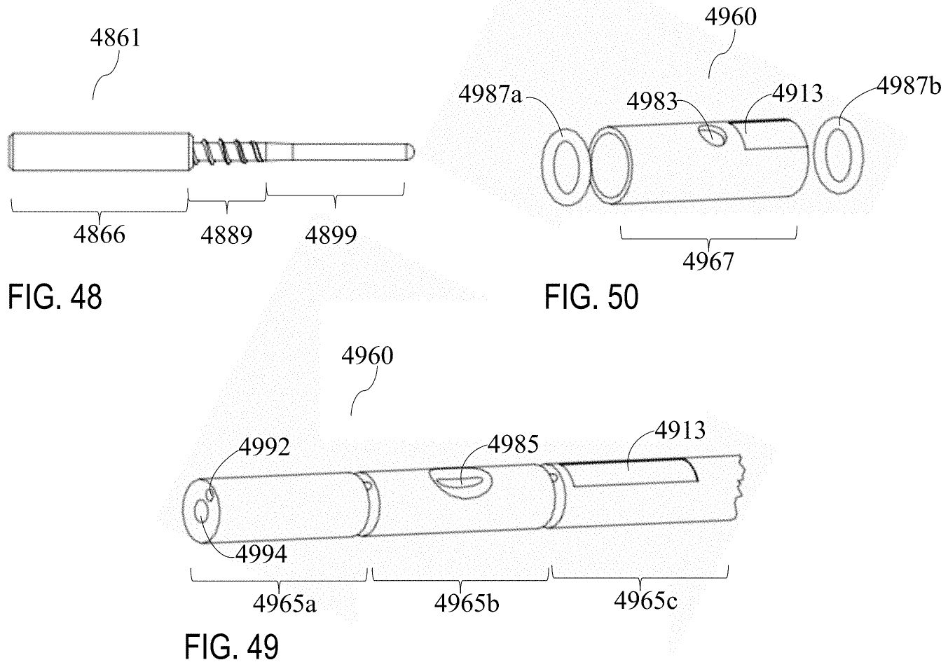

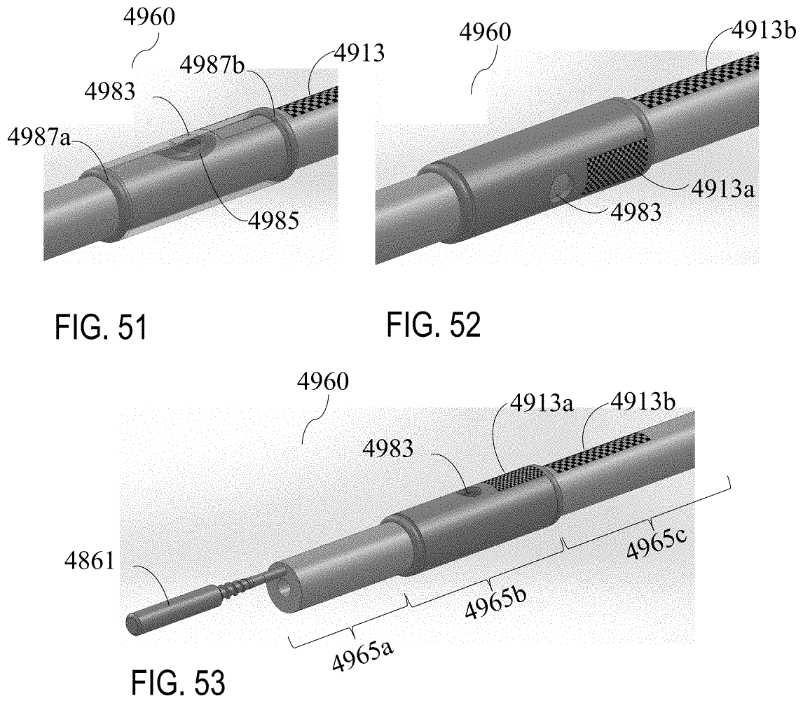

FIGS. 44 and 48-53 show one embodiment of a plug 4861 configured for insertion into any of the lumens of handle 4960 shown in FIGS. 49-53. For example, plug 4861 is insertable into guidewire lumen 4994 when infusion liquid is being passed through inflation lumen 4992; plug 4861 is insertable into the inflation lumen 4992 after the flexible member is expanded (e.g., prevents backflow of inflation fluid out of a proximal end of the assembly); plug 4861 is reversibly insertable into an inflation lumen 3992, 4492 until inflation is needed; in a three lumen handle, plug 4861 may occlude a lumen not in use; etc. Plug 4861 includes body 4866, coupled to an insertion section 4889 (e.g., tapered or not) coupled to a lead in section 4899, the insertion section 4889 and lead in section 4899 being insertable into a lumen, for example an infusion lumen, of elongate member or a handle attached to the elongate member. An embodiment of an infusion handle 4960 is shown in FIGS. 49-53. Handle 4960 includes an inner rotary body 4965 comprising a proximal section 4965a, a middle section 4965b, and a distal section 4965c. The middle section 4965b includes an inflation lumen access skive 4985 configured to inflate a flexible member when fluid is applied therethrough and is rotatably overlapped by outer rotary body 4967. Outer rotary body 4967 is coupled to a proximal sealing feature 4987a (e.g., O-ring) and a distal sealing feature 4987b (e.g., O-ring). Outer rotary body 4967 further defines an inflation aperture 4983. When fully assembled and in an open configuration, as shown in FIG. 51, an inflation lumen access skive 4985 of inner rotary body 4965 is aligned with an inflation aperture 4983 of outer rotary body 4967, such that liquid can be infused through the handle 4960 and through the flow reducing device (e.g., valve (e.g., tuohy borst) surrounding handle 4960 at aligned skive 4985 and aperture 4983; valve attached to proximal section 4965c, etc.). In a closed configuration, as shown in FIG. 52, an inflation lumen access skive 4985 of inner rotary body 4965 is misaligned or not aligned with an inflation aperture 4983 of outer rotary body 4967, such that fluid cannot be infused through the handle 4960 and through the flow reducing device into a flexible member. Handle 4960 may further include an extension indicator 4913a, 4913b to indicate when the skive 4985 and aperture 4983 are aligned (FIG. 53) and when the skive 4985 and aperture 4983 are not aligned (FIG. 52). Since the outer rotary body 4967 is rotatable relative to the inner rotary body 4965, a first or proximal end 4913a of extension indicator becomes aligned or misaligned with a second or distal end 4913b of extension indicator depending on whether the infusion lumen should be open or closed, respectively. Although handle 4960 was described with respect to FIGS. 39-49, it shall be appreciated that handle 4960 can be used with any of the flow reducing devices (e.g., FIGS. 12-25) described herein and/or with any of the other handle embodiments (e.g., FIG. 26-38) described elsewhere herein.

In some embodiments, the inflation lumen access skive 4985 further functions as an infusion lumen access skive 4985 to deliver liquid into the bodily lumen. In some such embodiments, a cover of the flexible member may include one or more perforations, such that liquid not only fills the flexible member but also fills the bodily lumen proximal to the flexible member.

In some embodiments, handle 4960 forms part of a proximal portion or end of an inner shaft of a flow reducing device such that handle 4960 and inner shaft are fully integrated and continuous. In some such embodiments, the inner rotary body 4965 is a specialized inner shaft and the outer rotary body 4967 is coupled to the inner shaft via a proximal sealing feature 4987a (e.g., O-ring) and a distal sealing feature 4987b (e.g., O-ring). In other embodiments, handle 4960 is couplable to a proximal portion or proximal end of an inner shaft of a flow reducing device such that the various lumens of handle 4960 and inner shaft are continuous and uninterrupted.

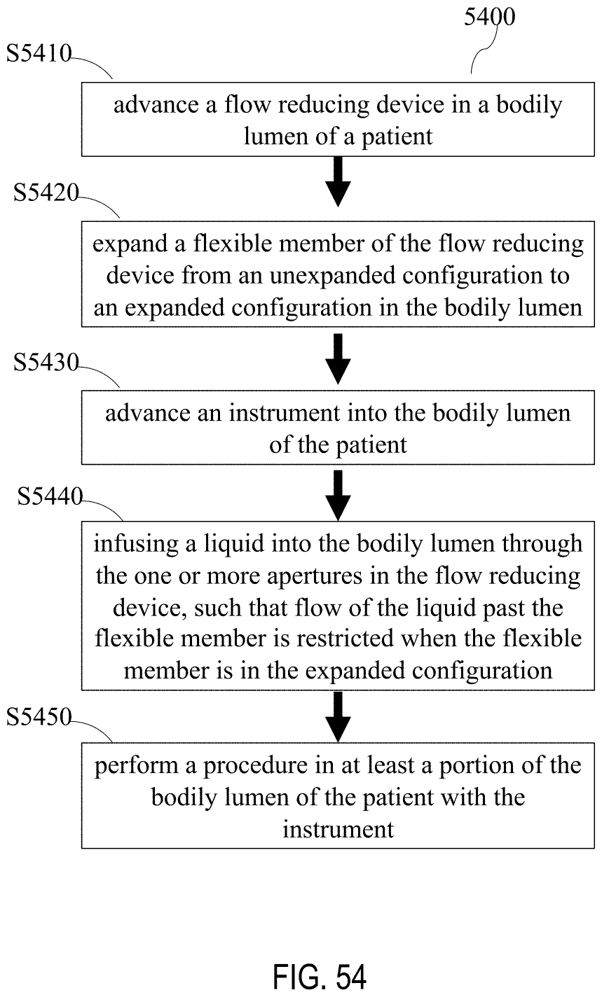

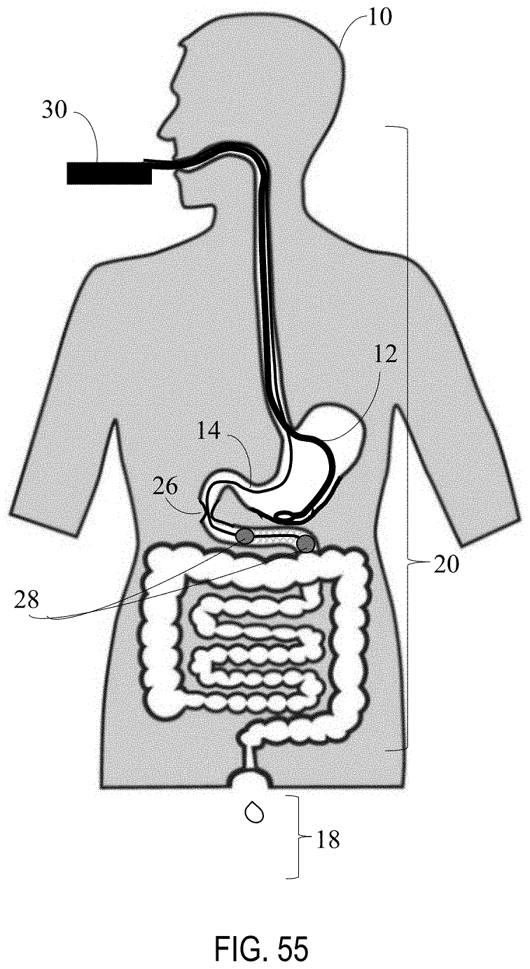

Now turning to FIG. 54, which shows a method 5400 of occluding at least a portion of a bodily lumen using any of the preceding embodiments of flow reducing devices and/or handles and/or infusion devices. Any of the steps of method 5400 may be used in any sequence and additional steps may be added or existing steps removed. A method 5400 for occluding at least a portion of a bodily lumen for or during a procedure includes advancing a flow reducing device through a bodily lumen, such that the flow reducing device comprises a flexible member and defines one or more apertures S5410; expanding the flexible member of the flow reducing device from an unexpanded configuration to an expanded configuration S5420; advancing an instrument into the bodily lumen of the patient 55430; infusing a liquid into the bodily lumen through the one or more apertures in the flow reducing device, such that flow of the liquid past the flexible member is restricted when the flexible member is in the expanded configuration 55440; and performing a procedure in at least a portion of the bodily lumen of the patient with the instrument 5450.

In any embodiments of the method 5400, any of the flow reducing devices described elsewhere herein may be used and/or employed. In any embodiments of the method 5400, an instrument includes any medical instrument, including but not limited to, ultrasound transducer or endoscope, ablation tool, biopsy tool, ligation tool, imaging tool (e.g., camera, microscope, ultrasound), sensor, stent, thrombectomy device, or any other medical device.

In some embodiments, method 5400 is particularly suited for performing an entero-enterostomy. In some such embodiments, block S5410 includes advancing a flow reducing device through a stricture in the GI tract of a patient, such that the flow reducing device includes a flexible member and defines one or more apertures. In some embodiments, block S5410 further includes positioning a distal end of an elongate member (e.g., endoscope, catheter, etc.) adjacent to a proximal side of a stricture in a GI tract of a patient; and advancing a flow reducing device through a lumen (e.g., working channel) defined by the elongate member (e.g., endoscope, catheter, etc.) and through the stricture in the GI tract.

In some embodiments, block S5420 further includes expanding the flexible member of the flow reducing device from an unexpanded configuration to an expanded configuration distal to the stricture in the GI tract.

In some embodiments, blocks 55430, 55440, and 55450 include advancing an echoendoscope into the GI tract of the patient; infusing a liquid into the GI tract through the one or more apertures in the flow reducing device, such that flow of the liquid past the flexible member is restricted when the flexible member is in the expanded configuration; and imaging at least a portion of the GI tract of the patient with the echoendoscope.

In some embodiments, infusing further includes coupling a liquid injection port (e.g., Tuohy borst valve) to a proximal end of the flow reducing device, such that the liquid injection port is configured to deliver the liquid through a lumen (e.g., infusion lumen) defined by the flow reducing device and out the one or more apertures of the flow reducing device into the GI tract, including one or more of: an esophagus, a stomach, a small intestine, or a large intestine.

In some embodiments, method 5400 includes positioning a distal end of an elongate member (e.g., endoscope, catheter, etc.) adjacent to a proximal side of a stricture in a GI tract of a patient. For example, for gastroenterostomy, the stricture is typically located in the proximal small bowel (duodenum), although could also be the distal stomach (called “gastric outlet obstruction” when contents back up into the stomach),

In some embodiments, the method 5400 further includes advancing a guidewire through the lumen of the elongate member and through the stricture of the GI tract of the patient, such that the flow reducing device is passed over the guidewire and through the stricture. A guidewire may be used when an endoscope and/or flow reducing device cannot be advanced pass the stricture.

In some embodiments, the method 5400 further includes removing the elongate member from the bodily lumen before advancing the instrument into the bodily lumen.

In some embodiments, method 5400 includes reducing a rate of flow of the liquid around the flexible member and through the downstream bodily lumen of the patient to less than about 300 ml/minute, less than about 230 ml/minute, less than about 200 ml/minute, about 200 to about 300 ml/minute, about 150 to about 250 ml/minute, about 100 to about 200 ml/minute, etc.

In some embodiments where the bodily lumen is the GI tract, the downstream GI tract includes one or more of: a small intestine, a large intestine, or a colon of the patient.

In some embodiments, method 5400 includes advancing an entero-enterostomy device through a lumen of the echoendoscope; and performing a entero-enterostomy procedure.

In some embodiments, method 5400 includes collapsing the expanded flexible member from the expanded configuration to the unexpanded configuration. In some such embodiments, collapsing may include moving an outer shaft proximally toward a proximal portion of an inner shaft to collapse the expanded flexible member from the expanded configuration to the unexpanded configuration. Alternatively, or additionally, suction or negative pressure may be applied to the infusion lumen (e.g., to remove fluid from an interior of the flexible member) to collapse the expanded flexible member to an unexpanded shape.

In some embodiments, method 5400 includes removing the flow reducing device from the GI tract. In the collapsed configuration (after the flexible member was expanded), a diameter of the flexible member may be greater than an unexpanded configuration before the flexible member was expanded. Such increased diameter may still be sufficiently small in diameter to be effectively removed from the bodily lumen.

In some embodiments, method 5400 includes attaching a handle to the flow reducing device to facilitate expansion or contraction or control of the flexible member. Any of the handles and/or infusion devices described elsewhere herein may be attached to any of the flow reducing devices described elsewhere herein. The method may further include actuating the handle to manipulate the outer shaft relative to the inner shaft. Such actuation may include moving a distal end of the handle that is coupled to an outer shaft toward a distal end of the device to expand the flexible member. Alternatively, actuating a handle may include rotating an outer rotary body relative to an inner rotary body to open an aperture for infusion of a liquid to expand a flexible member.

In some embodiments, method 5400 includes removing the echoendoscope from the GI tract.

In some embodiments, any of the systems and devices described herein may be used to prevent loss of a tissue specimen(s) that may migrate downstream with peristalsis after endoscopic resection (e.g., removal of a polyp in the duodenum). For example, the method may include deploying the flexible member through an elongate member (e.g., endoscope) under endoscopic guidance downstream from the lesion to be resected; removing the elongate member while leaving the flexible member in place; reinserting the elongate member alongside, adjacent to, or proximal to the flexible member; resecting a lesion to create a specimen with the elongate member; retrieving the specimen by pulling the flexible member proximally to “scoop” or “grab” or otherwise collect the specimen; collapsing the flexible member with the specimen secured therein; and removing the flexible member and elongate device from the patient.

As used in the description and claims, the singular form “a”, “an” and “the” include both singular and plural references unless the context clearly dictates otherwise. For example, the term “aperture” may include, and is contemplated to include, a plurality of apertures. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device or method.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure.

“Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A device configured to block at least a portion of a lumen of a gastrointestinal tract during an endoscopy procedure, the device comprising:
an inner shaft having a proximal portion, a distal portion, and defining a lumen therethrough and a first aperture; and
an outer shaft having a proximal end and a distal end coupled to the distal portion of the inner shaft, and defining a lumen therethrough, the outer shaft further comprising a flexible member and defining a second aperture positioned proximally on the outer shaft relative to the flexible member, wherein the inner shaft extends through the lumen of the outer shaft;
wherein at least a portion of the outer shaft is axially translatable toward and away from the distal portion of the inner shaft, and
wherein the flexible member is movable between an unexpanded configuration and an expanded configuration when at least a portion of the outer shaft is translated axially toward or away from the distal portion of the inner shaft.

2. (canceled)

3. The device of claim 1, wherein the flexible member is about 0.1 inches to about 5 inches from the distal end of the outer shaft.

4. (canceled)

5. The device of claim 1, wherein the inner shaft further comprises a stop configured to restrict movement of the outer shaft relative to the inner shaft and thus the flexible member.

6. The device of claim 1, wherein the inner shaft further comprises an extension indicator on a surface of the inner shaft that indicates an axial translation length required to move the outer shaft to expand the flexible member from the unexpanded configuration to the expanded configuration.

7. The device of claim 6, wherein the extension indicator further comprises a negative extension indicator indicating a state of over-expansion of the flexible member.

8. The device of claim 1, wherein the inner shaft further comprises a tip on the distal portion of the inner shaft, wherein the distal tip comprises a valve configured to prevent liquid from exiting the distal tip of inner shaft.

9. The device of claim 8, further comprising a liquid injection port coupled to the proximal portion of the inner shaft.

10. The device of claim 9, further comprising an infusion device coupled to the liquid injection port configured to deliver the liquid through the liquid injection port, through the first and second apertures, and into the gastrointestinal tract of the patient proximal to the flexible member.

11. The device of claim 1, wherein the flexible member comprises a proximal end and a distal end, wherein the proximal end of the flexible member is coupled to the outer shaft and the distal end of the flexible member is coupled to the inner shaft.

12. The device of claim 1, further comprising a handle having a proximal end coupled to the inner shaft and a distal end coupled to the outer shaft, such that the distal end of the handle is axially translatable to move the proximal end of the outer shaft toward and away from the distal portion of the inner shaft.

13. The device of claim 1, wherein at least a first half of the flexible member comprises a plurality of struts.

14. The device of claim 13, further comprising a cover configured to encase the plurality of struts.

15. The device of claim 13, wherein the plurality of struts encloses a filler material.

16. The device of claim 1, wherein the flexible member comprises a plurality of hydratable beads, such that the beads are configured to swell from an unexpanded state to an expanded state.

17. The device of claim 16, wherein the plurality of hydratable beads is configured to expand when a liquid is applied through the one or both of the inner shaft and the outer shaft.

18. The device of claim 1, wherein the flexible member comprises a braided material.

19. The device of claim 18, further comprising a cover configured to encase the braided material.

20. The device of claim 18, wherein the braided material encloses a filler material.

21. (canceled)

22. The device of claim 1, wherein the first aperture is substantially aligned with the second aperture when the flexible member is in the expanded configuration.

23. The device of claim 1, wherein the flexible member comprises a balloon.

24. The device of claim 1, wherein the flexible member is coated with an expandable material.

25. (canceled)

26. The device of claim 1, wherein the inner shaft and outer shaft are substantially rotationally fixed relative to one another.

27. (canceled)

28. A device configured to block at least a portion of a lumen of a gastrointestinal tract during an endoscopy procedure, the device comprising:
an elongate body having a proximal end and a distal end and defining a lumen therethrough, the elongate body further comprising a flexible member and defining at least two apertures, a first aperture positioned proximally on the elongate body relative to the flexible member and a second aperture being configured to inflate the flexible member;
wherein the flexible member is inflatable to an expanded configuration from an unexpanded configuration when a liquid flows through the lumen of the elongate body and out the second aperture of the elongate body.

29. The device of claim 28, wherein the elongate body further defines a second lumen configured to receive a guidewire therethrough.

30. The device of claim 28, wherein the first aperture is configured to deliver liquid into the gastrointestinal tract.

31.-49. (canceled)
